(19) United States
(12) Patent Application Publication
(10) Pub. No.: US 2014/0335617 A1
(43) Pub. Date: Nov. 13, 2014

(54) CONTROLLING SURFACE WETTABILITY OF ULTRAHIGH SURFACE AREA HIERARCHICAL SUPPORTS

(71) Applicant: Sharmila M. Mukhopadhyay, Beavercreek, OH (US)

(72) Inventor: Sharmila M. Mukhopadhyay, Beavercreek, OH (US)

(73) Assignee: WRIGHT STATE UNIVERSITY, Dayton, OH (US)

(21) Appl. No.: 14/277,205

(22) Filed: May 14, 2014

Related U.S. Application Data
(63) Continuation-in-part of application No. 13/681,752, filed on Nov. 20, 2012.

(51) Int. Cl. C23C 16/26; C23C 16/40; C12N 5/00

(52) U.S. Cl. CPC C23C 16/26; C12N 5/0068; C23C 16/402; C12N 2539/00
USPC 435/402; 428/143; 427/575

(57) ABSTRACT

The subject innovation is directed to hierarchical structures characterized by ultrahigh surface area and methods of fabricating the same, as well as attachment of functional species to these structures to alter interactions of these hierarchical structures with their environments, such as by making them permanently or reversibly hydrophilic. One such example hierarchical structure can include a solid substrate, an intermediate layer, at least one plurality of nanoscale attachments that are strongly bonded to the intermediate layer, and an oxygen containing species coating the at least one plurality of nanoscale attachments.

### CROSS-REFERENCE TO RELATED APPLICATION

This application is a Continuation-in-Part of pending U.S. patent application Ser. No. 13/681,752 (Atty. Dkt. No. WSU.P.1) entitled ‘ULTRAHIGH SURFACE AREA SUPPORTS FOR NANOMATERIAL ATTACHMENT’ and filed Nov. 20, 2012, which claims the benefit of U.S. Provisional Patent Application No. 61/563,152 (Atty. Dkt. No. WSU.P.US0001) entitled ‘ULTRAHIGH SURFACE AREA SUPPORTS FOR NANOMATERIAL ATTACHMENT’ and filed Nov. 23, 2011. The entireties of the above-noted applications are incorporated by reference herein.

### TECHNICAL FIELD

The subject innovation relates subject innovation to a hierarchical structure wherein a conventional compact or porous solid substrate is modified and further includes nanoscale attachments that significantly increase its surface area, as well as coatings on such nanoscale attachments to alter the properties of the nanoscale attachments, and provides advantages in surface-related applications such as catalysis, sensing, bioscaffolding, charge and gas storage, chemical, thermal and mechanical interactions.

### BACKGROUND

Prior patents have taught that carbon nanotubes and/or nanometer-scale metals can be grown using various methods. U.S. Pat. No. 7,148,512 discloses carbon nanotubes having nanometer-scale silver filling embedded on a silver colloid base to create a thermal interface for heat conduction in electronic components such as semiconductor chips. U.S. Pat. No. 8,207,658 discloses carbon nanotubes may be grown on metal surfaces, where the metal surface is preferably a nonmagnetic metal alloy, such as INCONEL 600. U.S. Pat. No. 7,438,885 teaches carbon nanotubes filled and decorated with palladium nanoparticles, wherein the synthesis involves arc-discharge technique with palladium chloride solution at temperatures greater than 3000° C. The palladium is ionized into nanoparticles and the graphite electrodes generate layers of graphene (carbon) which roll away from the anode and encapsulate or entrap the Pd-nanoparticles for use potentially as a gas sensor or as a means for hydrogen storage. U.S. Pat. No. 8,052,951 discloses bulk support media such as quartz fibers or particles, carbon fibers, or activated carbon, having carbon nanotubes formed therewith adjacent to metal catalyst species. Methods employ metal salt solutions such as iron chloride, aluminum chloride, or nickel chloride. The carbon nanotubes may be separated from the bulk support media and the metal catalyst species by using concentrated acids to oxidize the media and catalyst species.

Previously, nanomaterials such as nanotubes and nanoparticles that are not grown on metals or silicon wafers are either in the form of loose dust or entrapped in the pores. The resultant structures were then comprised of loosely combined units that cannot effectively restrain nanoparticles to prevent loss or leaching in service conditions.

Owing to the rise in demand for catalysis, sensing and environmental clean-up materials, there is a growing need for ultra-high surface area solids capable of more extensive interaction with the surrounding medium (air, water, biological fluids etc.). Two important parameters that influence these interactions are specific surface area (SSA) and interfacial energy. Materials with high surface area, such as high porosity solid structures and related articles discussed in connection with the subject innovation can provide for increased interaction with surrounding media for a variety of applications. One limitation is that the entire surface and related functionalities of graphitic materials may be under-utilized due to the hydrophobic nature of graphitic carbon surfaces, which may prevent effective contact with polar fluids such as water-based liquids. Graphite itself is non-polar and hydrophobic.

The subject innovation addresses these challenges by strongly anchoring nanoscale attachments onto surfaces of larger solids, thereby ensuring that the nanoscale attachments do not coalesce during transportation and storage and further ensuring they are not lost to the environment, thus preventing detrimental effects to the environment. The subject innovation synthesizes structures wherein attachment between nanoparticles and the parent substrate, including porous substrates, are at least as strong as the substrate itself. The subject innovation also provides for modification of these techniques and substrates to develop methods suitable for controlling the wettability of these hierarchical multi-scale carbon materials

### SUMMARY OF THE INVENTION

In at least one embodiment, a hierarchical structure characterized by ultrahigh surface area comprises: a solid substrate; an intermediate layer; at least one plurality of nanoscale attachments that are strongly bonded to the intermediate layer and a functional species coating the at least one plurality of nanoscale attachments.

In one or more embodiments, the hierarchical structure further includes a substrate surface, wherein at least one aspect of the substrate surface is modified to enhance bonding.

In one or more embodiments, the intermediate layer of the hierarchical structure is formed at the substrate surface.

In one or more embodiments, the hierarchical structure is characterized by a specific surface area of at least about 100 times the specific surface area of the solid substrate.

In one or more embodiments, the solid substrate of the hierarchical structure is compact or porous.

In one or more embodiments, the porous substrate comprises fibers, foams, sponges, fabric, paper, or combinations thereof.

In one or more embodiments, the substrate is inorganic or organic.

In one or more embodiments, the substrate comprises carbon, oxides, ceramics, glasses, polymers, or combinations thereof.

In one or more embodiments, the porous substrate is derived from plant, animal, or geological sources.

In one or more embodiments, the nanoscale attachments comprise nanotubes, nanoparticles, or combinations thereof.

In one or more embodiments, the nanotubes are cylindrical structures with diameters ranging from about 10 to about 50 nm and aspect ratios ranging from about 10 to about 100,000.

In one or more embodiments, the nanotubes are useful for guiding cell growth and for efficient thermal and electrical transport with the surrounding media.

In one or more embodiments, the nanoparticles comprise metals or compounds with surface-active properties suitable for catalysis, sensing, bioactivity, or antibacterial effect.

In one or more embodiments, the nanoparticles have a controlled particle size distribution from about 3 nm to about 8 nm.

In one or more embodiments, the nanoparticles have a broad particle size distribution from about 5 nm to about 100 nm.

In one or more embodiments, the nanoparticles are elements, compounds, or combinations thereof.

In one or more embodiments, the hierarchical structure is used for water purification.

In one or more embodiments, the hierarchical structure is used for sensing and remedial treatment of gases or fluids.

In one or more embodiments, a method of fabricating a hierarchical structure comprising: selecting and preparing a parent substrate, wherein the preparing may optionally include cleaning or activation; modifying the substrate surface to form an intermediate layer; attaching at least one plurality of nanoscale attachments, wherein the nanoscale attachments are selected from nanotubes, nanoparticles, or combinations thereof, onto the intermediate layer; optionally attaching a second plurality of nanoscale attachments, wherein the nanoscale attachments are selected from nanotubes, nanoparticles, or combinations thereof, onto the first plurality of nanoscale attachments and intermediate layer; wherein the steps of forming the intermediate layer and attaching the nanoscale attachments use one or more surface modification techniques selected from the group (a) wet chemistry, (b) chemical vapor deposition, (c) plasma deposition, (d) atomic layer deposition, (e) physical vapor deposition, (f) controlled environment heating, or a combination thereof; optionally heating in a controlled environment, and coating the nanoscale attachments with functional species (e.g., for affinity to various materials, such as polar fluids (e.g., via oxygen containing species, etc.), oils, proteins, etc.).

### BRIEF DESCRIPTION OF THE DRAWINGS

Aspects of the disclosure are understood from the following detailed description when read with the accompanying drawings. It will be appreciated that elements, structures, etc. of the drawings are not necessarily drawn to scale. Accordingly, the dimensions of the same may be arbitrarily increased or reduced for clarity of discussion, for example.

FIG. 1 illustrates a schematic diagram of the hierarchical structure in accordance with aspects of the innovation.

FIG. 2 illustrates a flow chart detailing the method of fabricating the hierarchical structure in accordance with aspects of the innovation.

FIGS. 3A, 3B, and 3C illustrate SEM micrographs of carbon nanotubes attached on commercial cellular foam using one of the processes in accordance with aspects of the innovation: (a) low magnification image, (b) higher magnification image of ligaments, and (c) image showing dense growth possible in some areas even inside a pore.

FIGS. 4A, 4B, and 4C illustrate SEM micrographs of carbon nanotubes attached on commercial reticulated carbon foam using the same process at (a) 50× magnification, (b) 1,000× magnification, and (c) 5,000× magnification.

FIGS. 5A and 5B illustrate a schematic profile of the surface of a pore showing that compared to a normal surface (a), the hierarchical surface with nanoscale attachments can accommodate significantly larger number of surface active nanoparticles as shown in (b). The increase in particle density can be controlled by density and length of nanotubes grafted on the surface.

FIG. 6 illustrates Electron Microscope images of Pd-NP (nanoparticle) structures in accordance with aspects of the innovation.

FIG. 7 illustrates SEM micrographs of silver nanoparticles attached to nanotubes that have been grafted on microcellular foam specimens.

FIG. 8 illustrates is an example of the particle distribution obtained for Ag nanoparticles obtained at two reduction temperatures.

FIGS. 9A and 9B illustrate shows how the hierarchical structures in accordance with aspects of the innovation are effective in bacteria removal from water. The dark spots are bacterial colonies formed in lake water when (a) untreated (b) treated with 4 mm×4 mm×2 mm samples of AgNP-CNT/Foam (as shown in FIG. 7).

FIGS. 10A and 10B illustrate schematics of example techniques by which the nanoparticle-nanotube activated porous material can be used to remove contaminants from liquids.

FIG. 11 illustrates ultrahigh surface area hierarchical substrates in accordance with aspects of the subject innovation, which can be very useful as supports for nanocatalysts, sensors and advanced composites.

FIG. 12 illustrates a method of applying an oxide coating to a nanotube-grafted substrate in accordance with aspects of the subject innovation.

FIG. 13 illustrates images of a carbon nanotube (CNT) hierarchical surface before and after silica coating by techniques of the subject innovation, and their respective responses to a drop of water.

FIG. 14 illustrates images of a water droplet on an initial CNT forest, after plasma treatment, and after reheating in air.

FIG. 15 illustrates microstructures of CNT-HOPG (highly oriented pyrolytic graphite), oxygen treated CNT-HOPG (O-CNT-HOPG) surface, and the same surface after air annealing, along with contact angles of water on each.

FIG. 16 illustrates C 1s scans of CNT-HOPG, O-CNT-HOPG, and air annealed-O-CNT-HOPG.

FIG. 17 illustrates the microstructure of silica coated CNT-HOPG surfaces (silica-CNT-HOPG).

FIG. 18 illustrates STEM images of both as-grown CNT and silica-CNT.

FIG. 19 illustrates XRD patterns of as-grown CNT and silica-CNT.

FIG. 20 illustrates the microstructure and contact angle of a silica-CNT-HOPG sample from experiments discussed herein.

FIG. 21 illustrates the average ΔP/L for a given volumetric flux over three samples each for bare reticulated carbon foam (RVC foam), CNT grafted RVC foam (CNT-RVC), and silica coated CNT-RVC (silica-CNT-RVC).

### DETAILED DESCRIPTION OF PREFERRED EMBODIMENTS

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

Embodiments of the subject innovation can be related to a hierarchical structure formed on a substrate that increases the specific surface area of the starting substrate by several orders of magnitude. The hierarchical structure of the subject innovation is referred to as an ultrahigh surface area material in that the surface area of the structure is at least several 100 times or more compared with that of the original substrate. The structure can comprise a solid substrate and a plurality of nanoscale attachments, wherein the substrate can be modified to ensure durability of the nanoscale attachments. The modified substrate can include an intermediate layer onto which at least one plurality of nanoscale attachments is attached. While the surface area of a solid substrate can be increased to a certain extent by other means such as porosity, there is a practical limit to such increase, because porosity makes the solid structurally weaker. Embodiments of the subject innovation can provide hierarchical structures wherein nanotubes can be attached on the solid, thereby providing orders of magnitude increase in surface area without compromising the strength or structural integrity, resulting in better performance while maintaining robustness.

Multi-scale hierarchical structures of the subject innovation can include solids that contain components characterized by different length scales. For example, nanoscale attachments according to the subject innovation can be strongly tethered to solid substrates, which are not characterized as nanoscale, such that the nanostructures do not detach from the parent substrate under service conditions. These structures can be suitable for anchoring nanoparticles, such as nanometals having known surface-active properties useful in non-limiting examples such as catalysis, sensing, hydrogen storage, electrochemical activity, cell interaction, or antimicrobial behavior.

Previously, nanomaterials with surfaces exposed to service environment have been mainly in the form of loose particles or dust that gets leached or dispersed into the environment (i.e. air, water, soil, or body) where used; thereby making nanomaterials expensive and risky to use. For example, previous investigators have entrapped nanoparticles in porous materials; however, the bonding between the nanotubes/nanoparticles has not been strong enough to effectively restrain nanoparticles to prevent loss or leaching.

Therefore, in conventional approaches, such nanoparticles having beneficial properties could not be used practically due to problems of retention of nanoparticles onto structure substrates leading to problems such as (i) loose nanoparticles clustering together during transportation and/or storage; (ii) nanoparticles escaping into the environment, thus requiring replenishment, i.e., redeposition; (iii) nanoparticles escaping into the environment thus causing potential detrimental effects on the ecosystem or other environmental concerns; and (iv) nanoparticles escaping into the environment thus causing unknown health risks to population due to nanoparticle exposure.

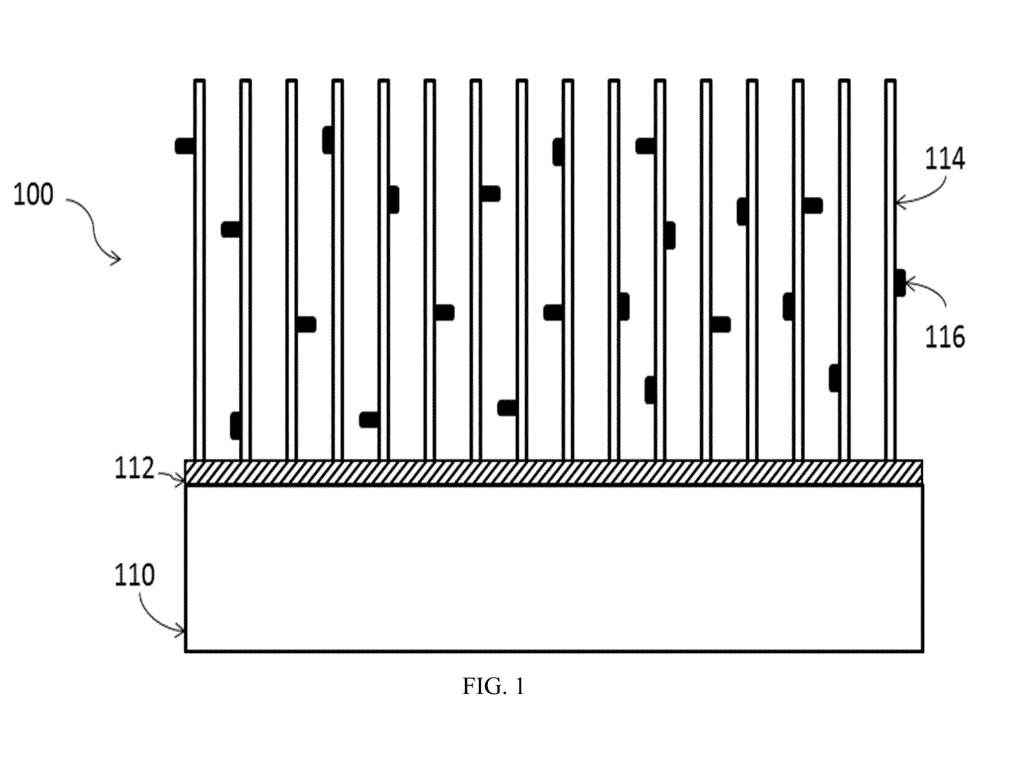

FIG. 1 is a schematic diagram of the hierarchical structures according to aspects of the subject innovation. Hierarchical structure 100 can comprise a solid substrate 110. The substrate can be modified or coated with chemicals, ions, plasma and/or heat to form an intermediate layer 112. To form the intermediate layer, one or more of the following non-limiting techniques may be used: (a) wet chemistry, (b) chemical vapor deposition, (c) plasma deposition, (d) atomic layer deposition, (e) physical vapor deposition, (f) controlled environment heating, or combinations thereof.

Further, nanoscale attachments such as nanotubes 114, nanoparticles 116, or combinations thereof can then be deposited or grown onto the intermediate layer 112 using liquid phase and/or vapor phase techniques involving one or more of (a) wet chemistry, (b) chemical vapor deposition, (c) plasma deposition, (d) atomic layer deposition, (e) physical vapor deposition, (f) controlled environment heating, or combinations thereof.

Different types of solids have been developed over the years for use in catalysts, sensors, filters, and sieves. Substrates 110 suitable for use in the subject innovation include both compact and porous solids. In at least one embodiment, the substrate may be inorganic or organic. In various embodiments, the substrate may include simple solids, fibers, foams, sponges, fabric, and/or paper. Substrates for use in one or more embodiments of the innovation may comprise carbon, oxides, polymers, or combinations or composites thereof. The substrate geometry may be simple or complex. Several grades of such material are already available from a wide range of commercial sources. These commercial substrates or other substrates can be advantageously engineered in the subject innovation to result in configurations wherein nanoparticles can be directly anchored to relatively flat or uneven porous solids; or alternatively, nanoparticles can be attached onto nanotubes that are tethered to the substrate; or combinations thereof.

In at least one embodiment of the subject innovation, the substrate can be highly oriented pyrolytic graphite (HOPG). In these or further embodiments of the subject innovation, the base structure can include microcellular or reticulate foam, an open cell structure, with porosities ranging from about 68% to about 94% and further characterized as having a high surface area.

Nanomaterials, materials having one or more dimensions less than 100 nm, are known for many beneficial properties. Nanomaterials may be referred to interchangeably herein as nanoscale attachments or nanoscale components. In at least one embodiment of the subject innovation, advantages arise from the increased specific surface area (SSA) of the solid without loss in mechanical integrity. Applications using structures comprising nanoscale attachments according to the subject innovation in at least one embodiment are directed to surface activity applications including: (i) catalysts for environmental purification using non-limiting examples of nanomaterials such as Pd, Ni, etc.; (ii) electrochemical activities such as charge storage and sensing using non-limiting examples of nanomaterials such as Pd, Ag, etc.; (iii) hydrogen storage and transport activities using non-limiting examples of carbon nanotubes (CNT) and Pd, etc.; (iv) antimicrobial activity using non-limiting examples of nanomaterials such as Ag, etc.; and (v) biological interactions such as cell scaffolding with non-limiting examples of CNT and/or anti-bacterial coatings using silver nanoparticles, etc. Using the advantageous structures comprising nanoscale attachments according to the subject innovation can result in time savings due to increased activity, space and weight savings due to miniaturization of the active device, and cost savings, especially for those devices utilizing precious metals.

In at least one embodiment of the subject innovation, the nanoscale attachments are nanotubes 114. In some embodiments, nanotubes according to the subject innovation may be carbon nanotubes (CNT), although in other embodiments, differing nanotubes can be used. In at least one embodiment of the subject innovation, CNTs can be grafted onto the base support. In yet another at least one embodiment of the subject innovation, the nanoscale attachments can be nanoparticles 116, which may include non-limiting examples such as iron nanoparticles (also referred to as FeNP), palladium nanoparticles (PdNP), silver nanoparticles (AgNP), or other nanoparticles. In at least one embodiment of the subject innovation, the nanoscale attachments can include nanoparticles attached onto nanotubes. In yet another embodiment, the nanoscale attachments can be further modified with chemicals or ions for fluid permeation and wettability.

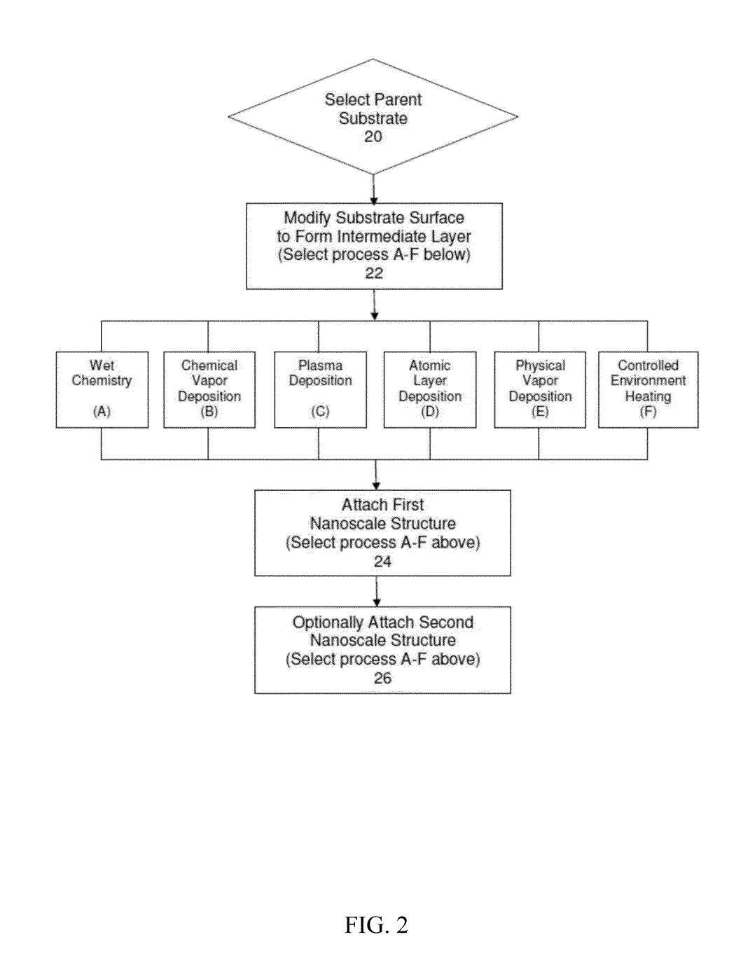

In at least one embodiment of the subject innovation, the nanoscale component(s) of selected metals can be attached to the substrate, wherein the substrate can be first modified at the surface to include an intermediate layer. The hierarchical structure of the subject innovation can be created by attachment of nanotubes onto substrates (e.g., commercially available substrates, etc.), which have been modified to include an intermediate layer. Strong attachment of the nanotubes and/or nanoparticles can be achieved by modifying the substrate to form an intermediate layer prior to attaching at least one plurality of nanoscale components. Examples of surface preparation/modification techniques according to the subject innovation can include, but are not limited to: cleaning and activation with chemical reagents, solvents, ions or plasma; deposition of nanoscale reactive layer by use of liquid precursors, molecular layering (atomic layer deposition), plasma deposition, chemical vapor deposition (CVD), or catalyst chemical vapor deposition (CCVD) methods; etc. In one or more embodiments of the subject innovation, surface modification techniques can be selected from the group (a) wet chemistry, (b) chemical vapor deposition, (c) plasma deposition, (d) atomic layer deposition, (e) physical vapor deposition, (f) controlled environment heating, or a combination thereof, as shown in FIG. 2. Technique selection can be tailored to the individual application, while also considering cost and quantity of the final commercial product. The surface preparation/attachment techniques according to the subject innovation can result in the formation of an intermediate layer 112.

In at least one embodiment of the subject innovation, the intermediate layer can be a nanometer-scale silica, also referred to as silicon dioxide or SiO₂, layer using microwave plasma deposition (MPD) of hexa-methyl-disiloxane (HMDSO) in O₂. Deposition of the oxide coating onto the substrate can be accomplished in three stages: (stage 1) introducing O₂ gas in to the microwave plasma chamber; (stage 2) subsequently flowing O₂ with HMDSO at increased microwave power to deposit oxide on the substrate surface; and (stage 3) introducing O₂ carrier gas into the chamber to stabilize the oxide coating at lower microwave power.

Silica thickness can be controlled by varying the coating time for stage 2 during MPD. The coating begins to manifest itself first as ‘island’ growth with heights of about 3 to 5 nm. The islands densely cover the surface of the substrate after about 30 seconds, and eventually coalesce by about 1 minute to form a uniform layer thickness of about 4 nm, with a surface roughness of less than 1 nm. The silica chemically bonds to the surface of the graphite substrate to form an interface of Si—C bonds. The intermediate layer of this particular embodiment, as defined herein, can include the Si—C bonds at the interface and silica deposited thereon.

After the intermediate oxide coating, or silica layer deposition as in this example, the sample can be transferred to the chemical vapor deposition (CVD) reactor for nanotube deposition by the floating catalyst method using a mixture of ferrocene and xylene. Longer CVD reaction times are possible and can result in further CNT growth and potential entanglement, which may be desired for some applications.

In at least one embodiment, the hierarchical structure including the substrate, the intermediate layer (oxide coating), and nanotubes deposited thereon can be further treated in order to include a second plurality of nanoscale attachments, for example, nanoparticles adhered onto the nanotubes. The nanoparticles, which may be gold, palladium, or silver, for example, can be formed by coating the hierarchical structure with precursor solution, reducing the metal containing precursors and subsequently heat treating in a controlled environment. These nanoparticles have the potential to act as catalysts, sensors, absorbents, or anti-microbial agents.

Similar or improved success may also be obtained by functionalizing the substrate to form an intermediate layer using one or more of the following techniques: (i) oxide coating using liquid precursors (e.g., commercial, etc.) followed by controlled thermal treatments, (ii) ultrathin coating of oxides such as aluminum oxide or silicon dioxide using atomic layer deposition, or (iii) intermediate layer of oxide using chemical vapor deposition (CVD). There are alternative means to prepare or modify the substrate surface in the practice of the subject innovation. For some substrates, as in one or more embodiments, the intermediate layer may be defined alternatively as an acid etched substrate surface.

Attachment of nanoscale components to the intermediate layer of hierarchical structures according to the subject innovation increases the available surface area of the overall solid by several orders of magnitude. This increased surface area can be used in multiple ways. One possibility is to use the bare nanotubes as “nano-radiators” that allow increased dissipation of heat or electric current across the surface. Another possibility is to use these hierarchical structures as a composite core material, or alternatively, as a template or scaffolding for cell growth. In one or more embodiments, the increased surface area can be utilized to support nanoparticles that can act as catalysts, sensors, or absorbents.

In at least one embodiment, the substrate with nanotubes, nanoparticles, or combinations thereof deposited thereon can be further functionalized for increased wettability or infiltration with other materials such as fluids, resins or energy storage (phase change) materials.

Strong attachment in a testing environment can be defined such that the nanoscale attachments do not get detached from the substrate before the substrate itself is internally damaged through the testing. As a practical matter, strong attachment ensures that desired performance of the hierarchical structure is met under use conditions.

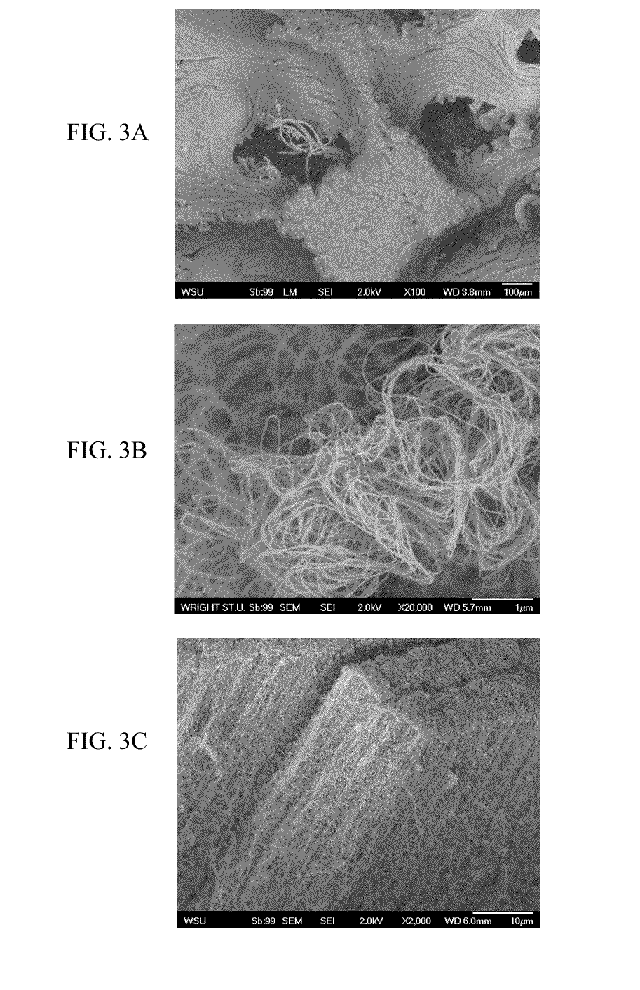

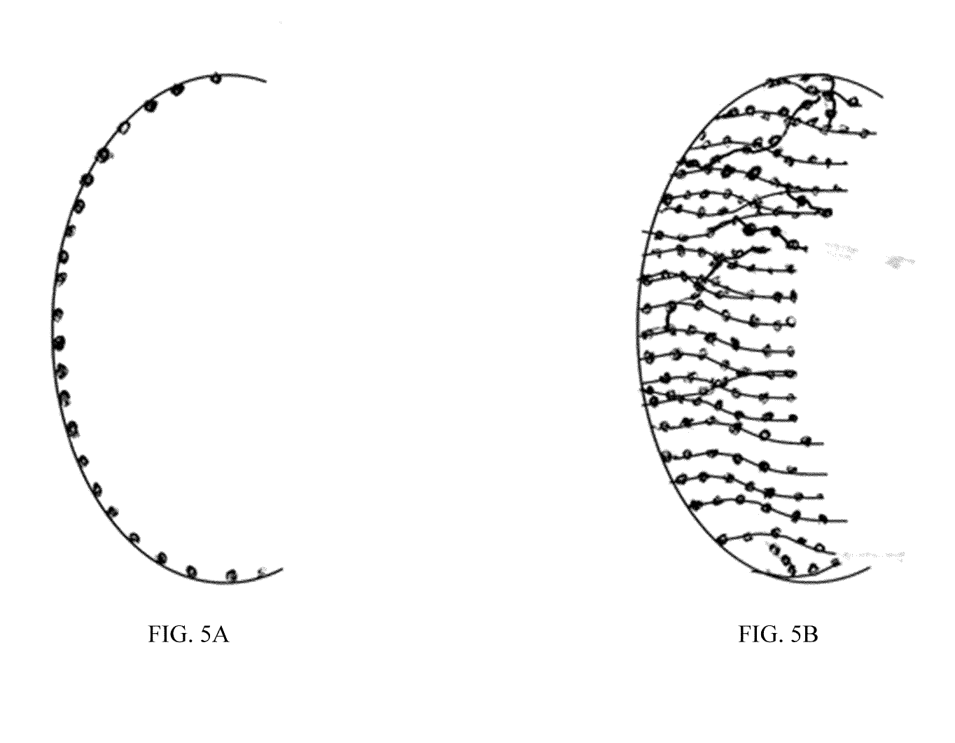

The initial success of nanotube and nanoparticle attachment can be observed by imaging with scanning electron microscopy (SEM), as seen in FIGS. 3, 4, 6 and 7. Measuring the average length, density and diameter of nanotubes provides an estimate of the increase in surface area. This number, along with a microscopic measurement of number of nanoparticles per unit length of nanotube, provides an estimate of the density of metal nanoparticles that can be packed on the surface. In one or more embodiments, over 7×10¹² nanoparticles/cm² (or 70,000 nanoparticles per square micron) could be accommodated on the surface using nanotube attachment as in FIG. 5B compared to 3×10¹⁰ nanoparticles/cm² (or 300 nanoparticles per square micron) on untreated surface similar to FIG. 5A.

To make direct determination of the specific surface area (SSA) of the substrate, the Brunauer-Emmett-Teller (BET) technique with nitrogen may be used. In one or more embodiments, the ultrahigh surface area hierarchical structures of the subject innovation are characterized by an increase in surface area by at least about 100-fold. In other words, the hierarchical structure can have a surface area about 100 times that of the original substrate. In other embodiments, the ultrahigh hierarchical structure can be characterized by having a specific surface area that is from at least about 100 to about 1000 times (10,000-100,000%) higher than the area of the starting substrate, while maintaining the same mechanical strength and minimal (less than 0.03 times or 3%) increase in weight.

In one or more embodiments, a starting foam substrate having an estimated specific surface area less than 0.02 m²/g could be modified with carbon nanotubes to create a hierarchical structure characterized by a surface area in the range of about 2 to about 4 m²/g.

In one or more embodiments, the hierarchical structures according to the subject innovation can be characterized by the nanoparticles having a controlled particle size distribution from about 3 nm to about 8 nm. In yet other embodiments of the subject innovation, the hierarchical structures can be characterized by the nanoparticles having a broad particle size distribution from about 5 nm to about 100 nm.

Qualitative failure analysis to determine the success of nanoscale attachment may be performed as follows. Samples broken at the edges can be analyzed microscopically to evaluate if the nanotube attachments, resembling nanotube ‘forests,’ are still attached. It has been observed in samples prepared according to the subject innovation that nanotubes remain attached, and fracture paths are indicated inside the graphite substrate itself rather than at the nanotube roots. This is defined as “strong bonding” of nanoscale attachments, when failure occurs in other parts of the substrate rather than at the intermediate layer wherein the nanoscale attachments are tethered to the substrate.

Another method useful in evaluating the success of nanotube attachment according to the subject innovation includes ultrasonication in water. The ultrasonication is believed to put large stresses at the base of the nanotube attachments, since their high aspect ratio would magnify any force at the tips caused by moving water. Despite that, failures observed indicate that the entire top layer of substrate peels off like a carpet; however, individual nanotubes (or nanoparticles attached to them) are not shed.

Yet another method to rapidly evaluate nanotube attachment is the Scotch® Tape test. Samples according to the subject innovation demonstrate that about 95% of the surface remains unchanged; in other words, nanoscale attachments are generally retained by the hierarchical structure. In the remaining about 5% area that does indicate loss of nanotubes, it has been observed that the entire outer substrate layer is removed like a carpet rather than as individual nanotubes. This thereby indicates failure occurring within the substrate itself, and is a further indicator of strong attachment of nanotubes to the substrate via the intermediate layer.

Samples according to the subject innovation have also been tested by rotation in water. These hierarchical structures have been attached to bottles filled with water and subjected to prolonged rotations for days and weeks at 32 revolutions per minute. No visible changes in nanotube or metal nanoparticle density after long exposures to rotation in water were observed using electron microscopy imaging techniques.

Shown in FIG. 2 is a flow chart of a method that can include the following steps in fabricating a multiscale hierarchical structure according to the subject innovation. While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

Step 20 includes selecting a substrate appropriate to the desired application. Step 22 includes modifying the substrate to form an intermediate layer using one or more surface modification techniques selected from the group (a) wet chemistry, (b) chemical vapor deposition, (c) plasma deposition, (d) atomic layer deposition, (e) physical vapor deposition, (f) controlled environment heating, or a combination thereof. Step 24 includes depositing or attaching at least one plurality of nanoscale attachments (nanotubes, nanoparticles, or combinations thereof) onto the intermediate layer, which may also be referred to as the coated substrate. The nanoscale attachments may be formed by using one or more techniques (a) through (f) described above or by combinations thereof. Step 26 includes optionally depositing or attaching a second plurality of nanoscale attachments (nanotubes, nanoparticles, or combinations thereof) onto the intermediate layer, the first plurality of nanoscale attachments, or to both the intermediate layer and the first plurality of nanoscale attachments. The nanoscale attachments may be formed by using one or more techniques (a) through (f) described above or by combinations thereof. Each of the steps 22, 24, and 26 may be optionally combined with heating in controlled environments such as in inert, oxidizing or reducing gases, or in vacuum as is known in the art.

In various aspects, embodiments of the subject innovation can be directed towards creation of hybrid hierarchical materials that consist of smaller nanoscale structures strongly tethered to larger solid substrates, and related methods of fabrication and use.

The hierarchical materials in accordance with aspects of the innovation are useful in applications such as, but not limited to, the following:

(i) thermal and charge dissipation structures made from carbon nanotubes attached to larger substrates: carbon nanotubes, having high electrical and thermal conductivity, can be used, for example, as nano-radiators for electronic cooling, electromagnetic interference shielding, encapsulation of energy storage materials, and other applications;

(ii) core structure for composites: the fractal geometry of the surface provides more intimate bonding with the matrix, thereby improving toughness and durability;

(iii) scaffolding for biological growth;

(iv) catalysts for electrochemical reaction, hydrogen storage and water de-contamination, utilizing nanoparticles such as palladium;

(v) antimicrobial agents and sensors, utilizing nanoparticles such as gold and silver. Solid silver attached to an anchor has several advantages over chlorinated compounds and other common anti-bacterial chemicals that are directly dispersed in the water, spread in the environment, and consumed in food. In addition to other side effects, these chemicals appear to increase the development of antibiotic-resistant strains of bacteria. Solids of the subject innovation can significantly reduce the need for antibacterial chemicals in the water and directly address some of the above issues.

Various modifications and alterations that do not depart from the scope and spirit of the innovation will become apparent to those skilled in the art. The innovation is not to be limited to the illustrative embodiments set forth herein.

What follows is a more detailed discussion of certain systems, methods, and apparatuses associated with aspects of the subject innovation. To aid in the understanding of aspects of the subject innovation, theoretical analysis and experimental results associated with specific experiments that were conducted are discussed herein. However, although for the purposes of obtaining the results discussed herein, specific choices were made as to the selection of various aspects of the experiments and associated setups—such as choice of materials, substrates, etc.—the systems and methods described herein can be employed in other contexts, as well. For example, various aspects of the subject innovation can be utilized in applications ranging from catalytic, environmental purification, charge or hydrogen storage, and petroleum cracking, as well as in other application. In some embodiments, different selections of materials, configurations, etc. can be selected than those used in the experiments discussed herein, and may have differing characteristics, as explained in greater detail herein.

### EXAMPLES

### Example 1

### Carbon Nanotubes on Silica Coated Graphite

Base substrates used were flat graphite as well as microcellular foams of graphitic carbon. These were modified by coating with a silica nano-layer using microwave plasma deposition (MPD) of hexa-methyl-disiloxane (HMDSO) in O₂. Deposition of the oxide coating intermediate layer was accomplished in stages: (stage 1) O₂ (99.99%) gas introduced into the microwave plasma chamber to clean the surface; (stage 2) O₂ and HMDSO introduced at microwave power of 250 W; and (stage 3) O₂ carrier gas introduced to stabilize the oxide.

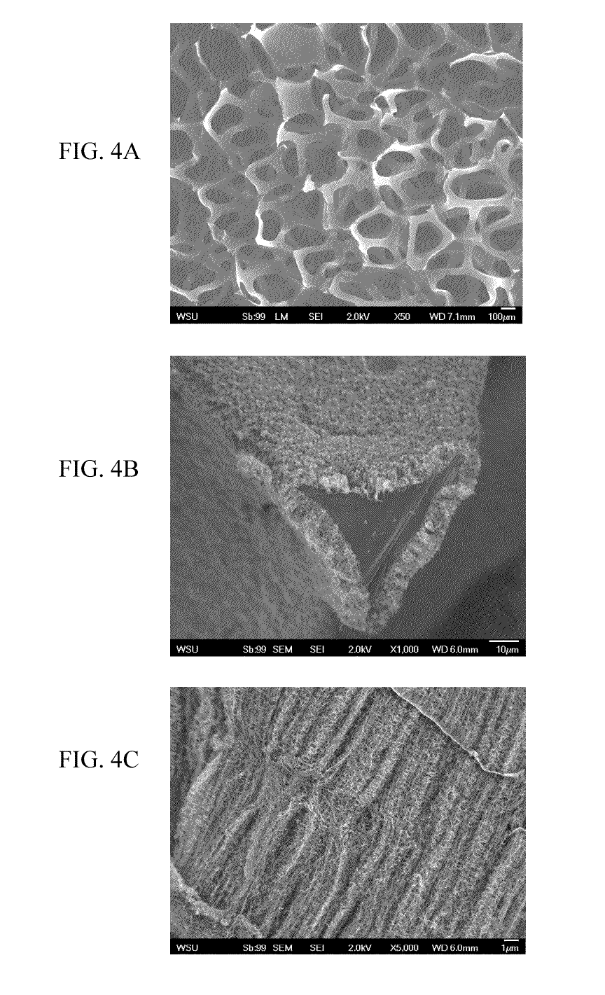

After silica layer deposition, samples were transferred to a CVD reactor for carbon nanotube (CNT) deposition. The CVD process parameters were optimized for purest nanotube growth. Optimization conditions depended upon the exact geometry of the substrate to be modified. The first stage of the CVD is used to heat the ferrocene/xylene solution prior to injection. The second stage of the CVD furnace is heated to the growth temperature for carbon nanotube fabrication (700-800° C.). Once samples were positioned in the reactor, a measured solution of ferrocene (C₁₀H₁₀Fe) dissolved in xylene (C₆H₄C₂H₆) was injected into a flowing gas mixture of argon and hydrogen. The substrates were heated to the growth temperature and subjected to deposition for specific times. After the allotted time, samples were allowed to cool in argon before removal from the CVD chamber. The deposition parameters for optimum nanotube morphology depended upon the initial geometry of the surface to be coated. FIG. 3 demonstrates a dense growth of carbon nanotubes is obtained on carbon foam as in this example using the surface modification technique of plasma pretreatment in combination with subsequent CVD deposition. The pretreatment level can be used to control the density of nanotubes grown on the surface, and the CVD process parameters can be adjusted for control of nanotube length and quality. FIG. 4 shows the growth of nanotubes using this method on reticulate carbon foam. FIG. 4A is a low magnification (50×) image of the foam as purchased. FIG. 4B (1,000×) shows that each strut can be covered with a dense carpet of nanotubes and FIG. 4C (5,000×) shows that the nanotube ‘forest’ can be very dense for long enough CVD times.

Semi-empirical predictions obtained by combining analytical modeling and micro-structural data indicate that it would be easy to obtain at least 100 times or more increase in surface area using the concepts of the subject innovation.

Direct specific surface area (SSA) measurements using the Brunauer-Emmett-Teller (BET) technique with nitrogen has been performed on cellular foam substrate material. It is seen that in commercial porous foams having a starting surface area of about 0.017 m²/g, the attachment of nanotubes results in a surface area of about 3 m²/g, implying an increase of well over 100-fold. Also significant, this occurs with negligible increase in volume and a weight gain of only about 2.5%. These advantages are anticipated to be significantly boosted for even longer grown nanotubes.

Thermal dissipation behavior of such structures into phase change materials has been tested and seen to be promising. The nanotube attachments are seen to increase the mechanical interlocking and prevent delamination between these solids and any matrix material, hence making them suitable for use in advanced composites.

These structures are also seen to offer accelerated growth of biological cells due to the scaffolding action of nanotubes on the surface. CNT-grafted foams show more prolific growth of healthy cells, which may result in faster bio-integration and healing of implants.

In addition to these advantages, at least one advantageous benefit of these hierarchical structures may come from the fact that the increased surface area can now harbor larger number of functional nanoparticles on its surface. FIG. 5 shows the schematic of the surface profile, indicating why a pore wall with attached nanotubes can support larger number of functional nanoparticles for applications such as catalysis, sensing, hydrogen storage, or antibacterial activity.

### Example 2

### CNT and PdNP on Graphitic Supports

Base structures tested were microcellular and reticulated carbon foam having uneven and irregular geometries as well as flat graphite substrates. Analytical reagents used, without further purification, were as follows: hexamethyl-di-siloxane (HMDSO), xylene, ferrocene, tetraamine palladium (II) nitrate solution (TAPN), methanol and concentrated nitric acid (HNO₃, 70%), and distilled water.

The as-received carbon foam substrate was modified, or pretreated, prior to attaching palladium nanoparticles by (1) nitric acid etching and (2) plasma assisted silica coating. Nitric acid etching was performed by immersing the carbon foam in 16M HNO₃ for a few minutes followed by sonication with distilled water to ensure complete removal of acid. Silica nanocoating was deposited onto the substrates in a microwave plasma reactor using HMDSO. The coating time was 15 minutes.

As an alternative to the above pre-treatments, some studies were also done on foam samples grafted with carbon nanotubes using the process discussed in Example 1. On all these samples, palladium nanoparticles (PdNP) were fabricated by liquid-phase synthesis technique followed by thermal reduction process. Tetraamine palladium nitrate (TAPN) was used as the metal precursor solution. Supports, cleaned with methanol and distilled water prior to infiltration, were immersed in the aqueous Pd precursor solution of tetraamine palladium (II) nitrate (TAPN) for specific period of time. The molar concentration used in this study was 62.5 mM TAPN and the supports were impregnated in the TAPN solution for 30 mins. The solid supports were recovered from the TAPN solution and the excess non-interacting solution on the sample was washed by briefly dipping it in methanol. The samples were placed on a ceramic boat and immediately transferred to the furnace for heat treatment processing. Heat treatment may include several individual steps, or using a combination of steps such as: drying, calcining, and reducing. In one example, the impregnated samples were dried at 100° C. for 12 hrs in the ambient atmosphere to eliminate water from the samples. Calcination was then carried out in either oxygen-rich ambient atmosphere (air) or oxygen-deficient inert atmosphere (Ar). The thermal profile in this step was controlled with a ramp rate of 10° C./min and held at 450° C. for 2 hrs. This process of controlled heating was adapted to avoid sintering. The final step involved thermal gas-phase reduction of Pd oxides to metallic Pd. In this example, the temperature was increased to 500° C. and held for 2 hrs using hydrogen gas (25 cc/min) as a reducing agent in an inert atmosphere of Ar (500 cc/min), Ar/H₂:20/1. The furnace was then allowed to cool down to room temperature in the reduced flow of Ar and H₂. Surface morphology of metallic nanoparticles and hierarchical structures were observed using JEOL 7401F Field Emission Scanning Electron Microscopy (FESEM). Statistical analysis was carried out on SEM micrographs using Scandium© SEM imaging software for JEOL 7401F FESEM.

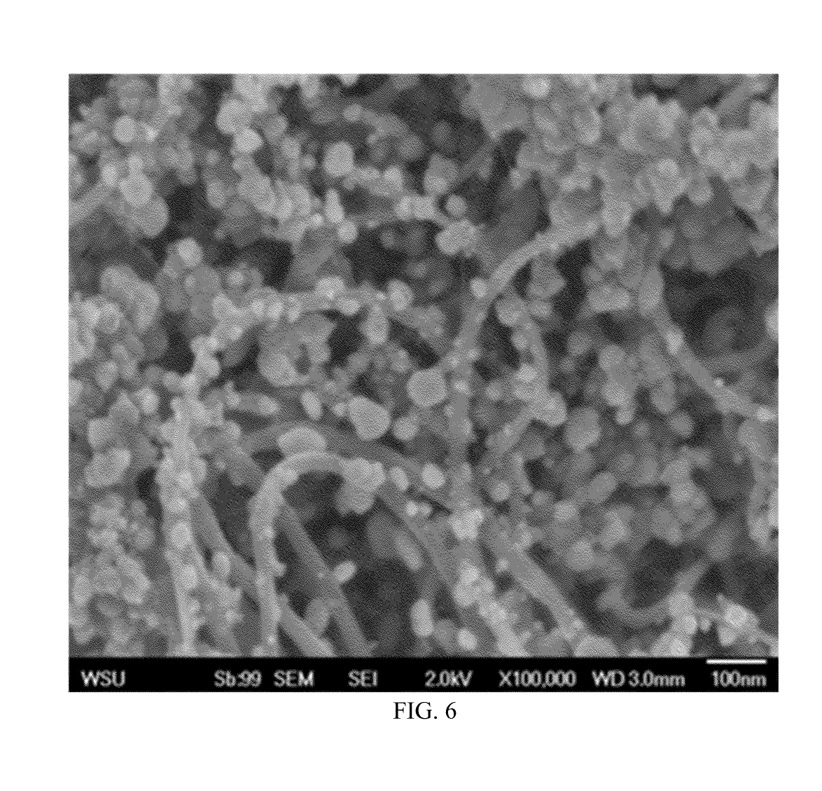

FIG. 6 demonstrates nanotubes attached to cellular carbon foam and subsequently functionalized with Pd nanoparticles.

### Example 3

### CNT and AgNP on Graphitic Supports

Several supports, or substrates, were tested for attachment of silver nanoparticles. They include (i) cellular carbon foam (ii) flat HOPG graphite (iii) carbon fibers and (iv) paper foils made of nanofibers, grapheme, and nanotubes.

Some of the samples were used “as-is,” while others were exposed to CVD to attach nanotubes as discussed in Example 1.

These structures were pre-wetted with methanol followed by dipping it in 0.24 M AgNO₃ solution for approximately for 1 hr. Samples were then placed on hot plate at 100° C. for 30 mins to remove moisture/water. These were finally reduced to metallic silver nanoparticles using the following process: DMSO has taken in a reduction beaker and a magnetic stirrer was placed at the bottom of the beaker. The sample was placed on a shelf above the stirrer. The reduction beaker was placed on a hot plate and heated to a temperature of from about 60° C. to about 80° C. followed by addition of 5 mg tri-sodium citrate. Continuous stirring helps rapid dissolution of citrate in DMSO. Silver nitrate coated sample was then placed on the sample shelf. After the desired time, samples were taken and washed off with distilled water and left to dry.

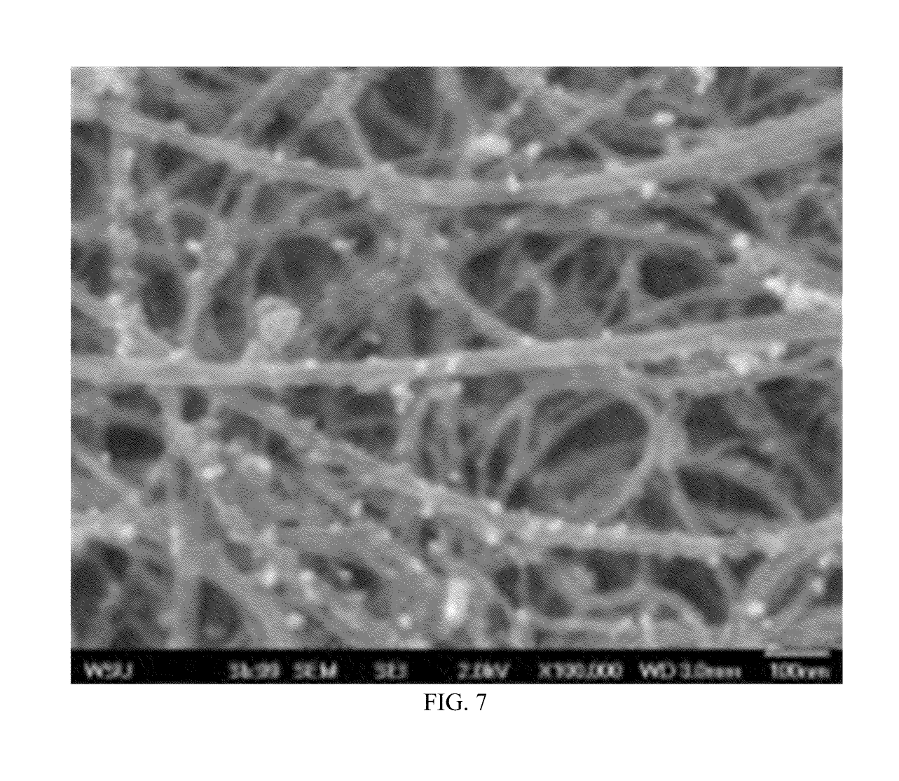

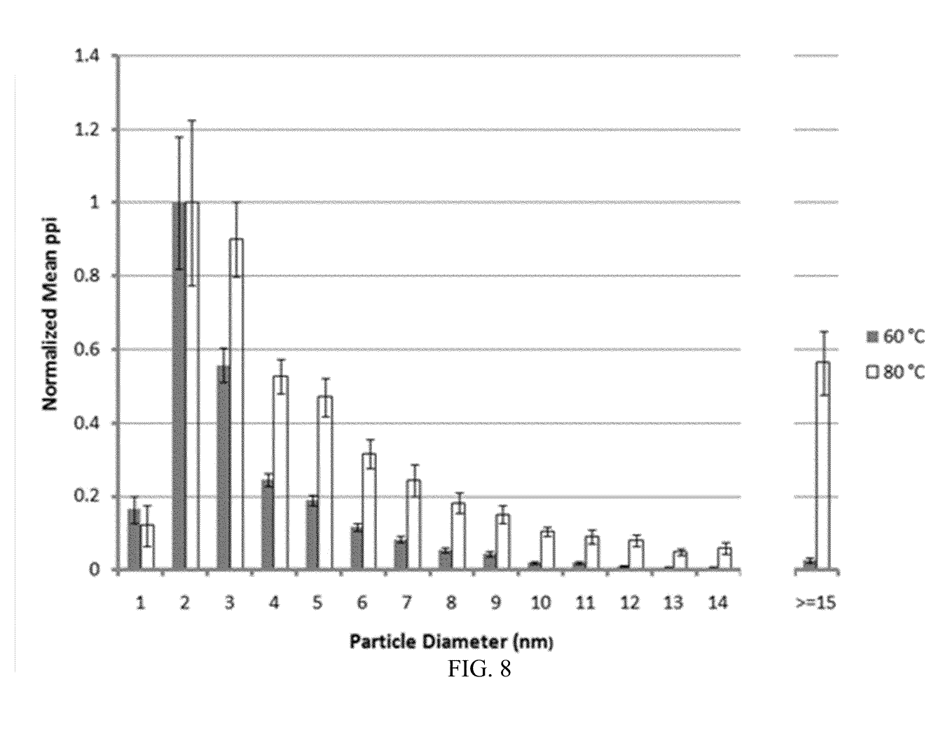

The particles were characterized using Field Emission SEM and detailed measurements of particle size distribution were made. FIG. 7 demonstrates nanotubes attached to carbon substrates, which are subsequently functionalized with silver nanoparticles (AgNP). FIG. 8 is an example of the particle distribution obtained for Ag nanoparticles obtained at two reduction temperatures. The distribution may also be altered to some extent with initial salt concentration.

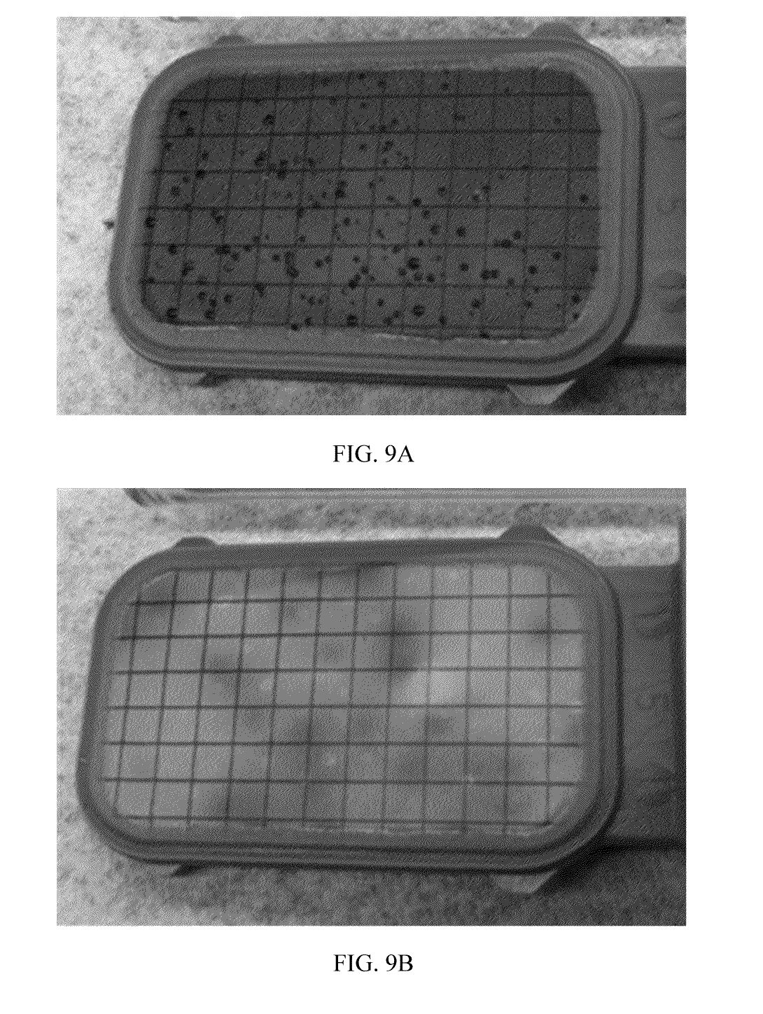

The anti-bacterial activity of these structures was measured with the idea of incorporating them into fluid purification systems. FIG. 9 shows the influence these materials have on lake water. FIG. 9A is the control, which shows a large number of bacterial colonies (dark spots) formed by incubation of untreated lake water. FIG. 9Bb shows results from identical testing performed on the lake water, after it was rotated in a 1-liter bottle containing 4 mm specimen of AgNP-decorated CNT-Foam support. The bacterial colonies are reduced drastically, or are non-existent.

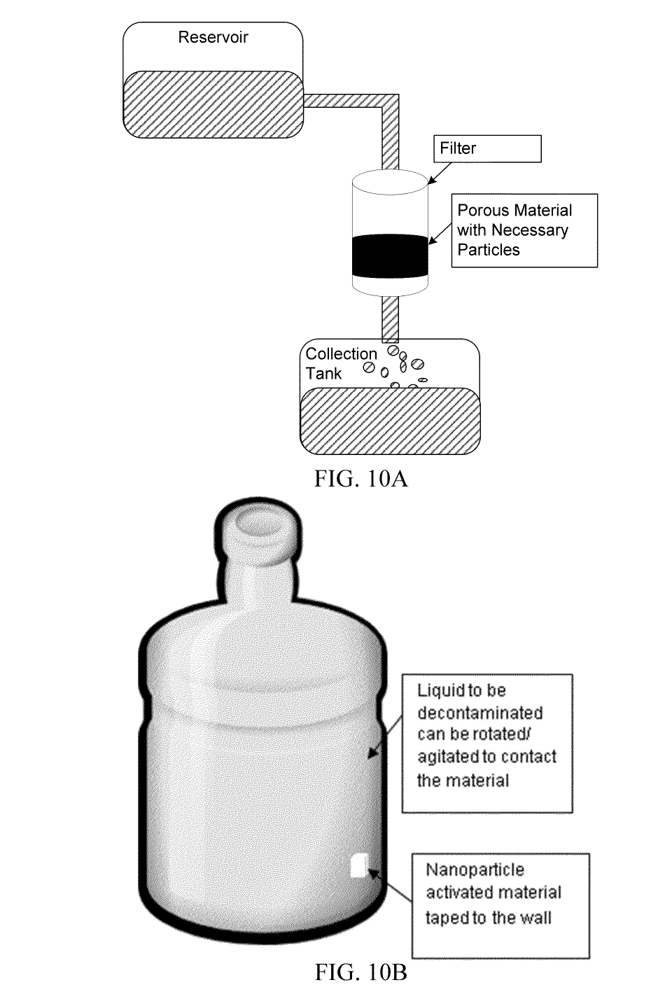

It is anticipated that the structures in this invention can be utilized in several different configurations for water purification, two of which have been tested successfully so far: flowing the contaminated water through the porous foam (as a filter shown in FIG. 10A), and rotation of Ag-NP decorated foam in 4000 times its volume of contaminated water (FIG. 10B).

### Example 4

### Hydrophilic Coatings to Enhance Functionality in Aqueous Environments

Carbon nanotube (CNT) grafted hierarchical surfaces offer high specific surface areas, a defining parameter for many applications involving solid-environment interactions. However, CNT grafted hierarchical surfaces tend to be super-hydrophobic due to their inherent physical and chemical characteristics. Control over surface wettability can play an important role in maximizing the surface-fluid interactions. In aspects of the subject innovation, two different types of surface modification techniques can be employed: techniques that can permanently make the surface hydrophilic, and techniques that can make the surface reversibly hydrophilic. Example techniques include dry etching via oxygen plasma to make the surface reversibly hydrophilic, and silica coatings via a liquid based sol-gel method to make the surface permanently hydrophilic. While plasma etching and heating could impart switchable wettability, silica coatings caused permanent wettability. Structure, morphology, composition and chemistry of these materials were investigated, and related to surface wettability and water flow. It was seen that plasma treatments did not alter the surface morphology in any way, but did change the chemical composition, which can be related to wettability. Sol-gel treatment coated the nanotubes with an amorphous layer of pure SiO₂. Water contact angle (CA) measurements discussed herein demonstrate that the plasma coating reduces contact angle, which can be subsequently restored by heat treatments. Silica coatings, on the other hand, impart permanent decrease in contact angle. Comparison of water flow through porous foams coated with CNT arrays and subject to similar treatments showed that hydrophilic coatings can significantly improve the flow along these surfaces due to increased wettability and capillary action. These results have important implications on many surface related applications of these materials such as catalytic supports, antimicrobial filters, microfluidic devices and environmental remediation.

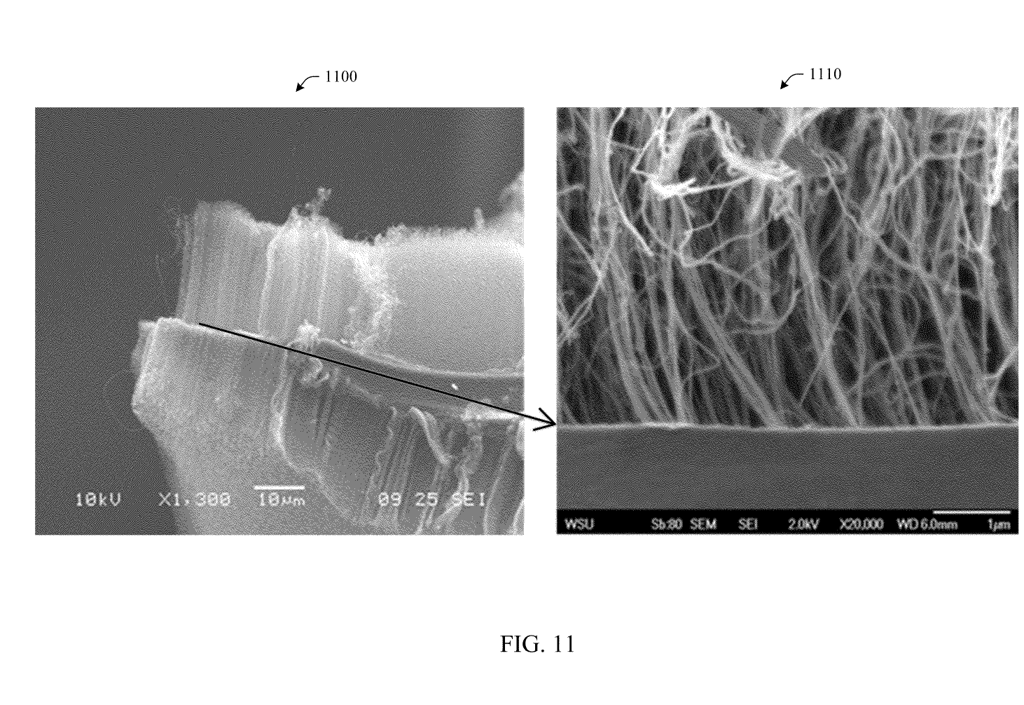

FIG. 11 illustrates ultrahigh surface area hierarchical substrates at 1100 and 1110, in accordance with aspects of the subject innovation, which can be very useful as supports for nanocatalysts, sensors and advanced composites. Many of the potential applications, such as water purification, catalysis etc., will involve flow and interaction of fluids through the nanotube carpet attached to their surfaces. Controlling the flow of aqueous fluids through this region can therefore be very useful.

In general, surface wettability of commonly known carbon-based structures such as graphite, carbon fibers, and isolated carbon nanotubes can be controlled by functionalization with oxygen-containing radicals via liquid-phase or gas-phase oxidation. Molecular groups such as hydroxyl, carbonyl, ester, and nitro groups can be introduced on the surface by liquid-based treatments with strong acids, peroxides, or gas-phase thermal or plasma treatments involving oxygen, Carbon dioxide, UV/Ozone radiation, air/water vapor and similar approaches. Multiple variations of one or more of these methods have been reported for conventional carbon materials by several investigators. In various embodiments, the subject innovation includes one or more techniques suitable for controlling the wettability of hierarchical multi-scale carbon materials such as those discussed herein and shown in FIG. 11.

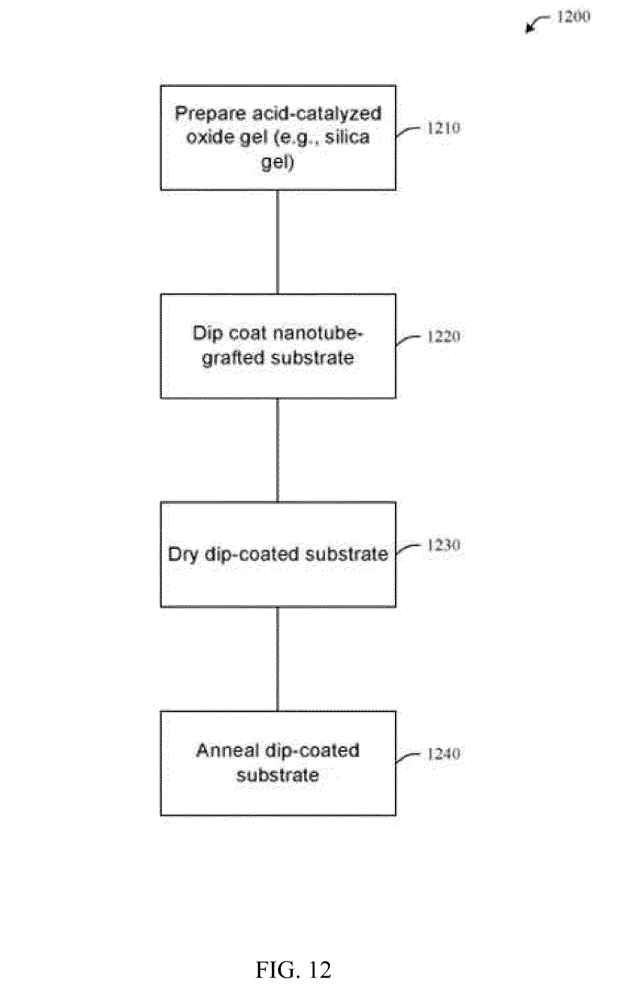

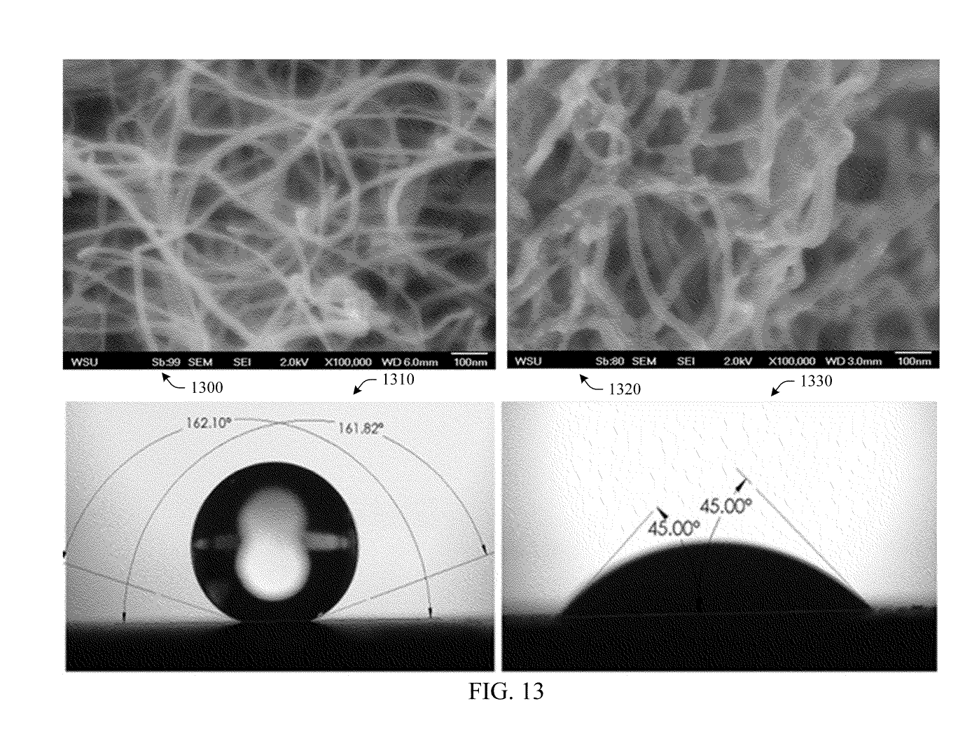

In permanently hydrophilic embodiments, this modification can include uniform coating of individual nanotubes in the carpet with a 1-3 nm layer of a polar oxide such as silica. This can be achieved by careful modification and optimization of a sol-gel process applied to traditional larger materials. FIG. 12 illustrates a method 1200 of applying an oxide coating to a nanotube-grafted substrate in accordance with aspects of the subject innovation. Method 1200 can involve preparing an acid catalyzed oxide gel (e.g., silica gel, etc.) at 1210, dip coating the nanotube-grafted substrate at 1220, drying (e.g., air drying, etc.) the substrate at 1230, and annealing (e.g, in inert atmosphere, etc.) the coated substrate at 1240. One difference between this method and a traditional sol-gel approach is the possibility of carefully optimizing the viscosity and permeability of the starting precursor solution (silicon containing molecule, alcohol and water with or without other solvents) that can enable precursor permeability and contact into the given CNT carpet having specific CNT densities and lengths. In this way, individual nanotubes in the carpet can be uniformly coated with the precursor before it starts gelling, so that clumps and flakes of oxide are avoided. Although only a few windows of precursor compositions are discussed herein, a spreadsheet recipe can be developed for different grades of application specific hierarchical substrates. FIG. 13 shows images of a CNT hierarchical surface before coating at 1300 and after silica coating by the technique of method 1200 at 1310, and their respective responses to a drop of water at 1320 (before treatment) and 1330 (after treatment), showing contact angles for water before and after treatment. It can be seen that individual nanotubes are coated with the silica at 1310, and that the water drop spreads more easily on the treated surface at 1330. Such treatments have been tested on porous carbon foams coated with nanotubes, and it has been seen that the flow of water, as well as the interaction of the water with nanotube walls (for the needed catalytic or antibacterial application), was significantly increased by such a treatment.

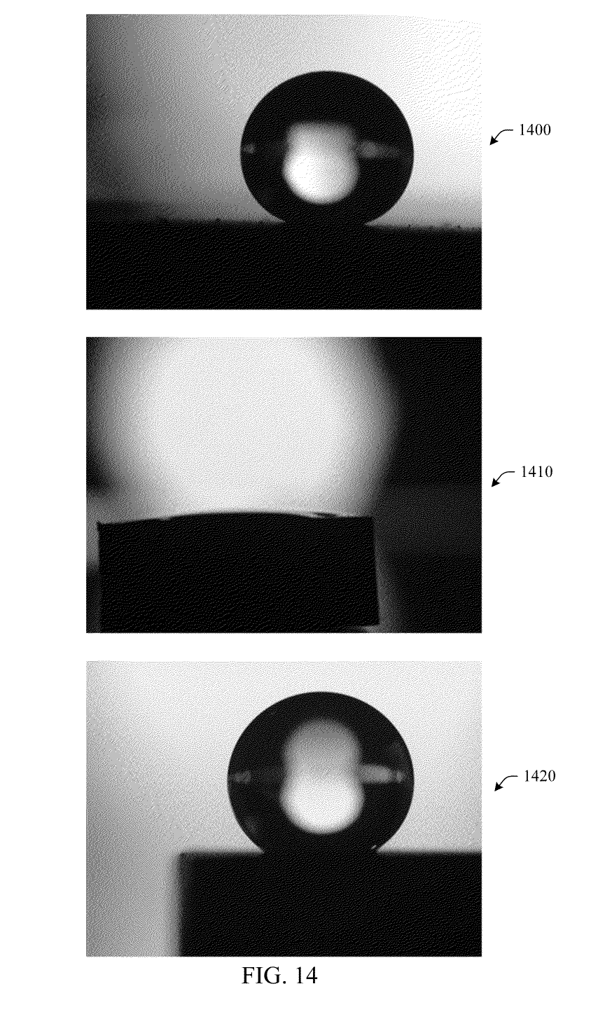

In plasma coating embodiments, plasma coating can be accomplished by treating the material in a low-medium power microwave plasma of oxygen gas for very short times (2-8 sec). An advantage of this approach is that this treatment is reversible, and hydrophobicity can be restored by simply heating the material in air at 100° C. This makes this low cost approach very suitable for applications where temporary wettability is desired. FIG. 14 shows images of a water droplet on an initial CNT forest at 1400, after plasma treatment at 1410, and after reheating in air at 1420.

In various embodiments, the subject innovation includes methods, compositions, and articles related to strategic control of the wettability of water and other polar fluids on ultra-high surface area hierarchical substrates. Control of wettability can be used to further enhance the properties and applicability of ultra-high surface area substrates. A well-aligned carpet of pure carbon nanotubes (CNT) on a porous substrate tends to be inherently hydrophobic in nature (it repels water away from the nanotube carpet). This hydrophobic nature can be attributed to both the surface chemistry of carbon nanotubes and to the hierarchical morphology of the CNT carpet. This hydrophobic nature may be useful for some applications, but not for others, which may need deeper permeation of water through the CNT forests. The latter applications will benefit if the CNT forests can be rendered hydrophilic. In various aspects, the subject innovation can employ either of two types of techniques for making the surface hydrophilic by attachment of oxygen containing species on the walls of the CNT, a first type of technique to make the surface permanently hydrophilic, or a second type of technique that can make the surface reversibly hydrophilic. In an example of the first type of technique, a liquid phase method of coating the CNT forest with a nanoscale oxide layer is described herein, which can make the surface permanently super-hydrophilic. In an example of the second type of technique, a dry plasma etch technique can be employed that can make the surface reversibly hydrophilic, wherein the hydrophilic behavior can be reversed by a simple heat treatment and reapplied when needed. This second type of technique can allow cycling of the material between hydrophilic and hydrophobic states. In additional embodiments, surface treatments can be applied to vary the interactions of nanostructures with their environment in other manners, such as to provide coatings with effects ranging from catalytic, photocatalytic, antimicrobial, petroleum cracking, environmental cleanup, alterations in oleophobicity, etc.

High porosity solid structures with strongly attached Carbon nanotube (CNT) arrays can offer many of the inherent nanoscale advantages while minimizing the environmental risks by anchoring them on robust, easy to handle, solids. Various estimates have shown that the SSA of these types of CNT-modified structures are easily several orders of magnitude higher than the starting porous solids. These multiscale morphologies have many applications in reinforcement for structural and thermal management composites, liquid purifications devices, tissue scaffolding and sensing. In addition, these materials offer excellent support for functional nanomaterials such as sensors, catalysts and disinfection devices.

According to wetting models for rough surfaces, water contact angle (CA) of inherently hydrophobic surfaces can be increased by “roughness factor” which makes CNT grafted surfaces super hydrophobic (≧1600). In various embodiments of the subject innovation, techniques can be employed that can improve the surface wettability of CNT grafted surfaces via dry and wet surface treatments.

Wettability is governed by both surface morphology and chemical composition. Surface chemistry of the CNT can be controlled through covalent functionalization by grafting oxygen-containing functional groups via liquid-phase or gas-phase “oxidative” treatments. In liquid phase methods, CNT can be treated with etchants such as nitric acid, sulfuric acid, mixtures of both and/or “piranha” (sulfuric acid-hydrogen peroxide). In gas phase oxidation, CNT can be treated with oxygen (thermal or plasma), carbon dioxide, UV/ozone radiation, or with air/water vapor at high temperatures. While many of these methods increase surface wettability by grafting oxygen-based functional groups, many can destroy the CNT structure itself causing damage. In accordance with certain aspects of the subject innovation, CNT grafted surfaces (or other articles with nanotubes grafted onto a substrate in accordance with aspects of the innovation described herein) can be made reversibly hydrophilic, such as by gently treated with oxygen microwave plasma at room temperature, which can improve the wettability without damage. In this case, the chemical species can be weakly physisorbed, and can be removed by heat treatment. While this approach allows cycling between hydrophobic and hydrophilic behavior, permanent hydrophilicity can also be achieved in accordance with other aspects of the subject innovation. One such technique can involve coating the nanoscale structure with a polar oxide such as silica.

Thin film coatings that completely coat nanotube surfaces without clumping are complicated by their small dimensions. This becomes more challenging when the nanotubes are in the form of arrays on a larger surface, and hence involve a multi-scale solid geometry. In experiments discussed herein, a permanent change in hydrophilicity was induced via a simple solution based sol-gel method for silica coatings, although it is to be understood that examples herein are provided solely for the purposes of illustration, and in various aspects of the subject innovation, similar techniques can be used with other coatings or decorations, which can provide characteristics usable in a range of applications. For example, other oxide or metallic coatings can be employed, such as metal oxides, for example, titanium oxide (e.g., in photocatalytic applications, etc.), mixed metal oxides (e.g., as charge storage devices, etc.), silver (e.g., antibacterial applications, etc.), palladium metal or oxide (e.g., for hydrogen storage, removal of contaminants, petroleum cracking, etc.), etc. The sol gel method can deposit a silica layer on isolated CNT powders. In aspects of the subject innovation, this process can be modified to get uniform or coating of hydrophilic silica (or other uniform or particulate coatings, such as the examples discussed herein, etc.) on the hierarchical solid consisting of CNT-grafted solid substrates (or on substrates with other grafted nanotubes, etc.). These can be equally effective for CNT arrays on planar solids such as graphite as well as on high porosity fabric or foams of any material capable of withstanding temperatures in excess of around 600° C., including but not limited to ceramics, metals, carbon, etc.

Surface morphology and chemical compositions were studied using field emission electron microscope (FESEM) and X-ray photoelectron spectroscopy (XPS) respectively. Crystal was studied using combination of transmission microscope pictures and X-ray diffraction (XRD).

Increased wettability can enable efficient utilization of the extensive surface area offered by the CNT-grafted structures, and also can enhance capillary fluid flow through the CNT carpets. These have important implications for several applications that will benefit from these solids. Experiments discussed herein examined the influence of these surface treatments on wettability and fluid flow. The former was monitored through contact angle measurements, and the latter by pressure drop (ΔP) vs. fluid flux (q) characteristics.

Wettability Experiments

Two kinds of support structures were used in the experiments: highly oriented pyrolytic graphite (HOPG) and reticulated vitreous carbon foam (RVC) [. The HOPG was a standard flat carbon substrate having well defined geometry, hence used for surface chemistry and wettability studies. The RVC foam, with its interconnected open cell morphology, was used for studying the fluid flow behavior with identical surface modifications.

For the sol-gel process, tetraethyl orthosilicate was used as silica precursor [TEOS; Si(OCH₂CH₃)₄, Si—OEt (Alfaa aesar; 98%)], distilled water (H₂O) for hydrolysis, and absolute ethyl alcohol (EtOH) as common solvent for TEOS and water, and hydrochloric acid (HCl) as acid catalyst for hydrolysis and condensation reactions.

The carbon nanotube grafting was a two-step process; deposition of silicon oxide (SiOₓ) buffer layer, followed by CNT deposition via thermal CVD method using xylene and ferrocene as carbon and catalyst sources respectively. The details of CNT grafting on the selected structures are discussed above. Carbon nanotubes were grafted on both selected support structures.

For surface etching in this experiment, 99.9% pure oxygen gas was used as precursor gas. Carbon nanotube grafted HOPG (CNT-HOPG) surfaces were placed in microwave plasma reactor operating at 115 W. Oxygen gas was fed into vacuum chamber and ionized using microwaves and etched CNT-HOPG surface for 10 seconds.

In reversibly hydrophilic experiments, the hydrophobicity of the CNT-HOPG surface was restored via air annealing at 110° C. for 1 hr.

Silica coating on CNT grafted carbon structure as performed in the example experiments involved multiple steps: acid catalyzed silica gel preparation, silica coating on CNT-carbon via dip coating method. Reaction chemistry of acid catalyzed sol-gel polymerization of TEOS in EtOH and H₂O has been well studied in conventional applications. In this process, silica precursor solution was prepared by mixing TEOS, EtOH, and H₂O in the volumetric ratios of x:1:1 respectively. Keeping the EtOH to H₂O ratio to 1:1, varying concentrations of TEOS to H₂O ratio; 1:2, 1:8, 1:30, and 1:50 were tried to optimize the silica layer thickness on CNT-carbon. The initial mixtures were mixed thoroughly for 1 h followed by adjusting the pH to 3.0 by adding HCl. The solution was further mixed for a 1 h until it became colorless and clear. CNT-carbon structures were soaked in the prepared silica precursor solution for 1 h and followed by air drying for 12 hr. Further drying was carried out on hot plate for 12 h at 100° C. Finally, the samples were annealed at 5000° C. at a rate of 20° C./min in Argon (Ar) atmosphere and kept there for 2 h before cooling them down to room temperature at a rate of 2° C./min. Unlike annealing in air as used in some conventional applications, Ar atmosphere was used to prevent oxidation of CNT and HOPG support.

The morphology, surface chemistry, and crystal structure, of silica-CNT-carbon were characterized using Field emission scanning electron microscope (FESEM), scanning transmission electron microscope (STEM), X-ray photoelectron spectroscopy (XPS) and X-ray Diffraction (XRD) respectively. Microstructure characterization of silica coated CNT-carbon were done using FESEM and STEM. For STEM analysis, silica coated CNT were peeled off from the carbon substrate and loaded on the TEM grids. Crystallographic orientation of synthesized AgNP on carbon structures were studied using X-ray mini diffractometer, MD-10, using a monochromoatized Cu Kα radiation at 25 kV and 0.4 mA. Surface chemistry of processed silica thin film was characterized using XPS system from Kratos (Axis Ultra) using mono-chromatized Al Kα (1486.6 eV) as X-ray source. CASA software was used for spectrum analysis and processing. The carbon is peak at 284.5 eV was taken as a reference position, a well-established value for HOPG, for charge correction.

A simplified in-house built goniometer was used for measuring contact angles (CA). In this set-up, a camera equipped with magnifying lens was mounted on a tripod while the sample is placed on a fixed sample stage. The surfaces were air brushed to remove dust particles and 5 μl of deionized water was gently placed on the sample surface using a micropipette. A box with double window, each on either side of the sample, was placed over the sample. One window was covered with diffuser paper to reduce the light intensity, generate a homogeneous dark background, and minimize reflections at water drop. Finally, the camera height and the distance between camera and the sample were adjusted to precisely determine position of the triple line, the intersection of solid-liquid-air interfaces.

Pictures were loaded in SolidWorks for processing. To measure the CA, a baseline was manually selected by choosing two points (intersecting points of drop profile meeting the flat surface) to define the baseline and three points along the drop profile. Tangents to the profiles were drawn from the meeting point of the profile to the base line and CA was calculated by measuring the angle between tangent and baseline. To improve precision, multiple measurements on multiple drops were obtained using this method and averages and standard deviations reported.

The behavior of fluid interaction with these surfaces was studied by attaching CNT arrays (and subjecting them to identical surface treatments) on porous RVC foam samples that could be inserted in a cylindrical water flow cartridge. Pressure drop per unit length (ΔP/L) across these samples were measured as a function of water flow velocity (θ). Assuming the fluid flow through porous materials is incompressible, ΔP vs. θ graphs were obtained for all the concerned porous structures. In this experiment, water flow rate was controlled by a master flux L/S (model 7518-10) controller. The filtration system included a porous structure fitted inside acrylic casing. Fluid in the storage container (water in this experiment) was fed into the porous material using a pump drive. The storage container, pump drive, filtration system, and collecting tank all were connected in series via ⅛ inch latex tubing. A manometer was connected at the fluid entry side while leaving the exit end open to the atmosphere. The height difference (Δh) in manometer “U” column was measured and the pressure drop (ΔP=ρ×g×Δh) was calculated, where ρ is the fluid density, and g is the gravitational constant. Superficial fluid velocity (θ) or volumetric flux (q) is (vol. flow rate (Q)/cross sectional area (A)), and was taken on the X-axis. Fluid volume flow rate was varied for specified intervals and pressure drop per length (ΔP/L) was measured at each flow rate.

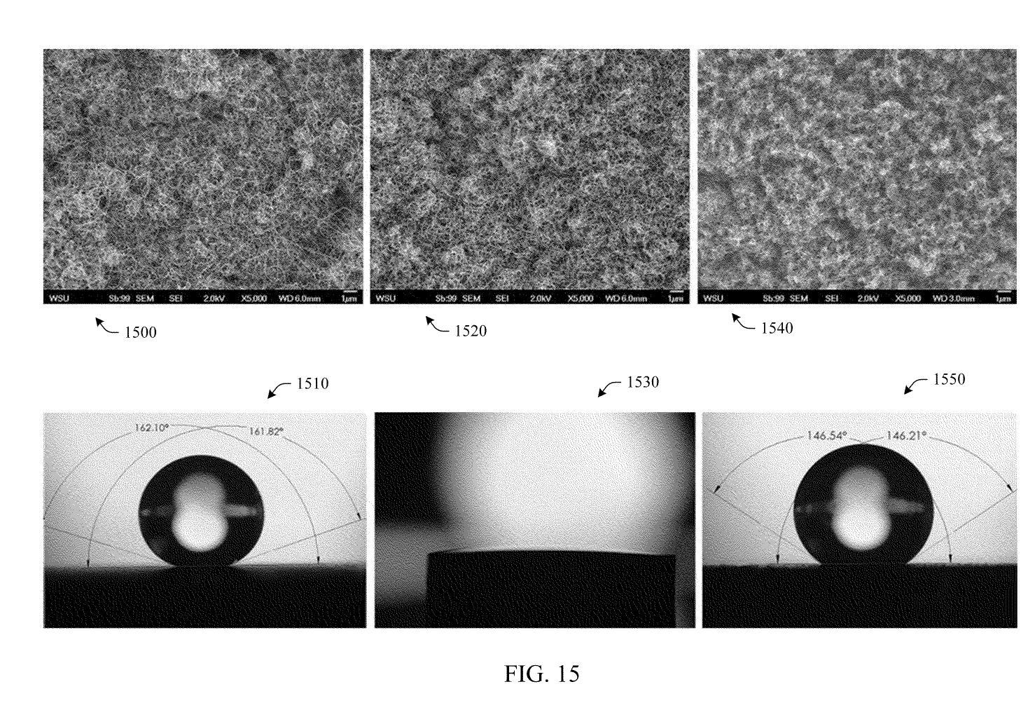

In the plasma oxygen experiment, carbon nanotube arrays grafted on HOPG substrate (CNT-HOPG) were used as the base-line surface and fabricated as explained above, and surface wettability was quantified as contact angle (CA) and measured as described earlier. Wettability of any surface is governed by both morphological structure and chemistry. The CNT-HOPG surface was treated with 115 W oxygen plasma for 10 sec as explained above. Care was taken, since dry etching with oxygen and ozone plasmas have been reported to cause structural damage to CNT due to defect creation and rupture of the hexagonal network. In connection with these experiments, this effect was observed at higher exposure times and microwave powers, but not at the operating microwave power and exposure time discussed herein. FIG. 15 shows microstructures of CNT-HOPG at 1500 and oxygen treated CNT-HOPG (O-CNT-HOPG) surface at 1510, and it can be seen that there was no noticeable change in morphology. It can be seen that CA was drastically decreased, from 162.5±2° before treatment, as seen at 1520, to less than 10° with 10 sec oxygen treatment, as seen at 1530. When CA is too low, water easily seeps into the CNT carpet, and quantitative accuracy is questionable, hence only the largest measurable water drop is shown at 1530. The conversion from super-hydrophobic to hydrophilic surface is clear.

Air annealing of O-CNT-HOPG sample was performed as explained above, and this surface (air-O-CNT-HOPG) showed no visible microstructural changes, as seen at 1540. However, hydrophobicity of CNT grafted surface was restored as evident from CA measurement. The measured CA on air-O-CNT-HOPG surface was 150±10°, as seen in 1550. Earlier studies that have treated loose CNT with oxygen and ozone plasmas have shown reversal of wettability after high temperature vacuum annealing or hydrogen treatments. These experiments have been able to create hydrophilic surface with very gentle room temperature microwave plasma that can be reversed at relatively mild temperature in air, making the process significantly more scalable. In fact, this hydrophilic-hydrophobic treatment could be repeated on the same sample multiple times, indicating that it does not compromise the sample structure in any way.

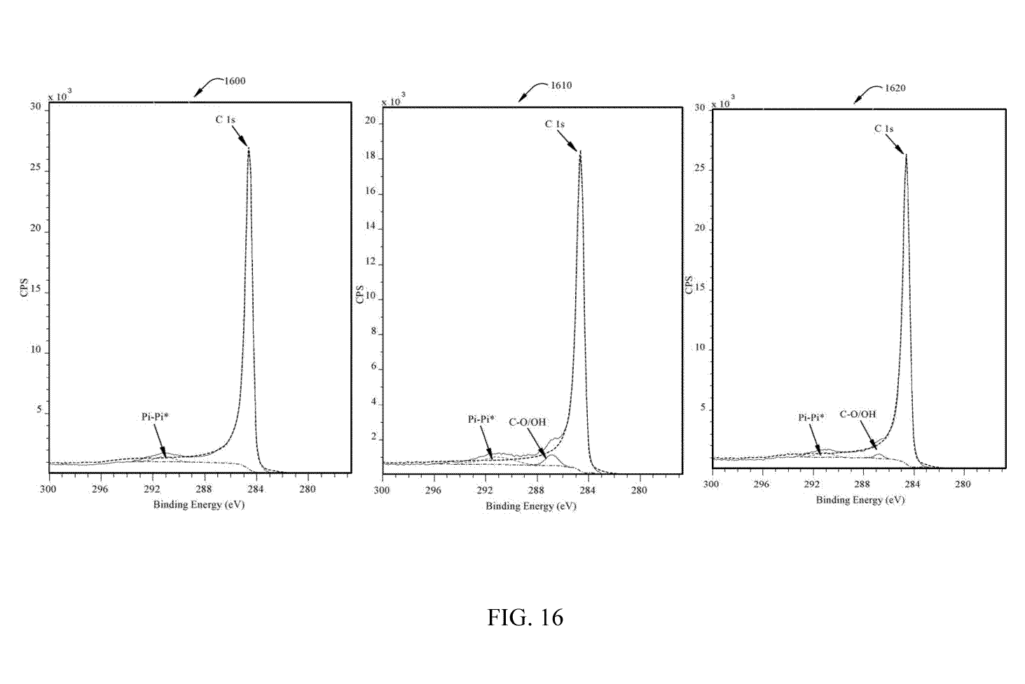

Influence of surface chemistry on wettability of surface modified CNT-HOPG was studied using XPS. FIG. 16 illustrates the peak analysis of CNT-HOPG, O-CNT-HOPG, and air annealed-O-CNT-HOPG, at 1600, 1610, and 1620, respectively. The deconvoluted peak positions and their corresponding concentrations, averaged over two regions, are shown in Table 1, below, showing the influence of the C/O ratio on contact angle and component based quantification. It is clear that oxygenated carbon component was introduced with plasma oxygen treatment, and was significantly reduced with air annealing. Conversely, the corresponding CA was decreased to less than 10° with oxygen etching and increased by to 146° with annealing. From this analysis, it can be seen that the oxygenated carbon species were closely related to wettability while physisorbed oxygen (included in the total O1s peak) is not a reliable indicator. In the experiments, it was seen that hydrophilic behavior created by microwave oxygen plasma can easily be reverted to hydrophobicity with simple heating, unlike earlier studies with RF plasma and Oxygen ozone etched processes, which seem to require high temperature heating in vacuum or hydrogen. The easy transition between super hydrophobic to super hydrophilic can have important applications, such as self-cleaning surface, hydro dynamic skin friction reduction, drug delivery and fluid separation devices. On the flip side, if applications require sustained hydrophilic behavior even at higher temperatures, this approach may not be a suitable one. Such situations will need a technique for more permanent surface modification, as discussed below.

TABLE 1
Influence of C 1s photoelectron peak components on contact angle

| | | | | Air |
|---|---|---|---|---|
| | | | Oxygen - | annealed- |
| | | CNT-HOPG | CNT-HOPG | Oxygen- |
| | BE positions | (%), | (%) | CNT-HOPG |
| | (eV) | θ = 164 ± 2° | θ = ≦10° | θ = 150 ± 2° |
| C asymmetric | 284.6 | 97.7 | 92.1 | 97.2 |
| peak | | | | |
| C—O/OH | 286.6 ± 0.1 | below | 3.0 | 0.9 |
| (hydroxyl) | | detection | | |
| π → π* shake | 291 ± 0.1 | 2.3 | 4.9 | 1.9 |
| up peaks | | | | |

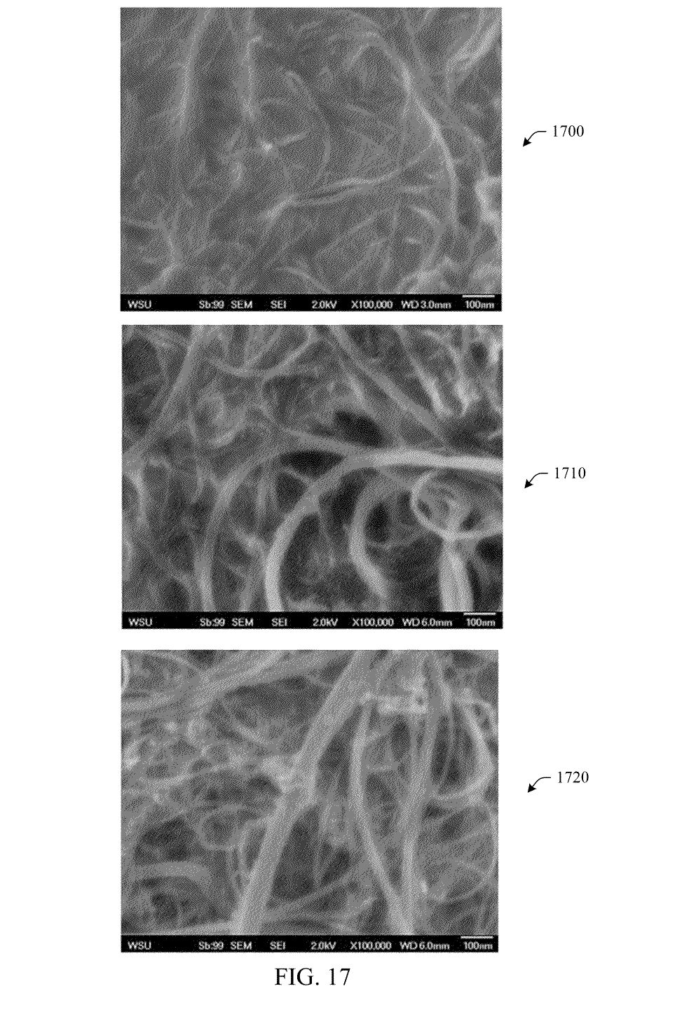

In a polar oxide coating experiment, CNT-HOPG surfaces were dip-coated with varying ratios of sol-gel solution as explained above. FIG. 17 illustrates the microstructure of silica coated CNT-HOPG surfaces (silica-CNT-HOPG). It can be seen that a concentrated silica solution resulted in silica clumping, more like a silica-CNT composite microstructure, as seen at 1700. However, as the concentration of TEOS was decreased from 1700 (TEOS to water ratio of 1:8) to 1710 (ratio of 1:33) and to 1720 (ratio of 1:50), silica thickness was controllable to the thickness scales suitable for CNT surfaces. At the concentration ratio of 1:50, individual CNT morphology was retained with a thin layer of coating, as seen in 1720. This was selected as a final recipe for the remainder of the experiment. It is to be understood that although this specific ratio was used in the experiments, in various aspects, different ratios can be used, for example, between about 1:40 and about 1:60, between about 1:30 and about 1:70, etc. Additionally, these ratios may depend on other characteristics, such as materials selected for nanotubes, coatings, etc. For example, simpler surface with sharper and more sparse nanotubes may benefit from higher ratios than 1:50, such as around 1:30 or higher, where more complex surfaces with longer and denser nanotubes may benefit from lower ratios, such as around 1:70 or lower, and longer process times.

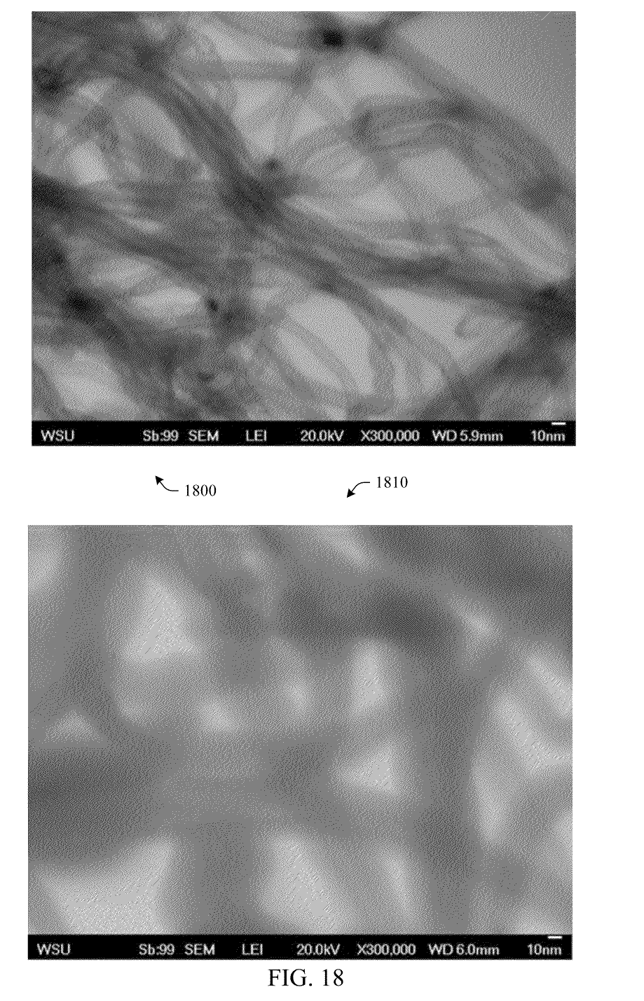

Transmission mode microstructures were taken using a STEM detector. For STEM analysis, a layer of silica coated CNT/graphite was peeled off and loaded on copper grids. FIG. 18 illustrates the STEM images of both as-grown CNT and silica-CNT in 1800 and 1810, respectively. In 1800, the thin walls of CNT and hollow space can be clearly seen. The average size of CNT outer and inner diameter was 15 nm and 10 nm respectively. In the case of silica coated CNT, a fuzzy and texture-less layer was seen on the CNT. Silica grown by sol-gel is expected to be amorphous without any long range order. The thickness of this region varied greatly and few thickly coated regions were observed. In various aspects not pursued in the experiments, one or more surfactants can be added to the sol-gel precursor in order to obtain more uniform silica layers. However, it can be seen from these results that the CNTs were coated with an additional film.

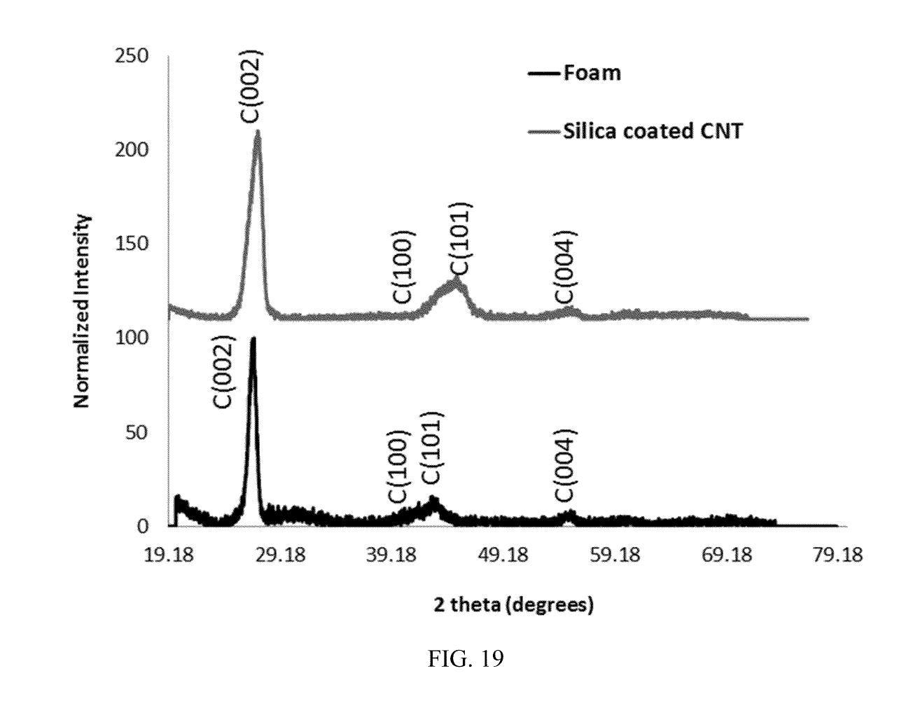

Further analysis was done to study the crystal structure of silica coated CNT-carbon surface. From the XRD patterns shown in FIG. 19 it can be seen that the diffraction peaks for both as grown CNT and silica-CNT were exactly identical and related to graphite and CNT only. The absence of any diffraction peaks from the presence of silica indicated that the formed silica was 100% amorphous. This result was in agreement with previous studies related to silica films prepared on carbon/graphite via this method.

For surface chemistry analysis of the silica film, detailed XPS analyses was performed on baseline CNT-HOPG and silica-CNT-HOPG. From this analysis, it was observed that the silica coated surfaces were free of reaction byproducts and contaminants. The region based semi quantification studies are shown in Table 2, below.

TABLE 2
Region based semi quantification CNT-HOPG and silica-CNT-HOPG

| | | |
|---|---|---|
| BE (eV) | FWHM | At % |

CNT-HOPG

| | | | |
|---|---|---|---|
| C 1s | 284.6 | 0.655 | 98.97 |
| O 1s | 533.2 | 2.048 | 1.03 |

Silica-CNT-HOPG

| | | | |
|---|---|---|---|
| C 1s | 284.6 | 0.643 | 68.75 |
| O 1s | 533 | 1.71 | 21.07 |
| Si 2p | 103.9 | 1.676 | 10.18 |

Upon silica coating, there was no observable difference either in binding energy (BE) and shape of C is peak is before and after silica coating. From detailed analysis of peak positions and shapes, and comparing them with peaks from standard silica films as well as quartz glass, it was concluded that formed silica was chemically identical to pure silicon dioxide (Si⁴⁺/SiO₂).

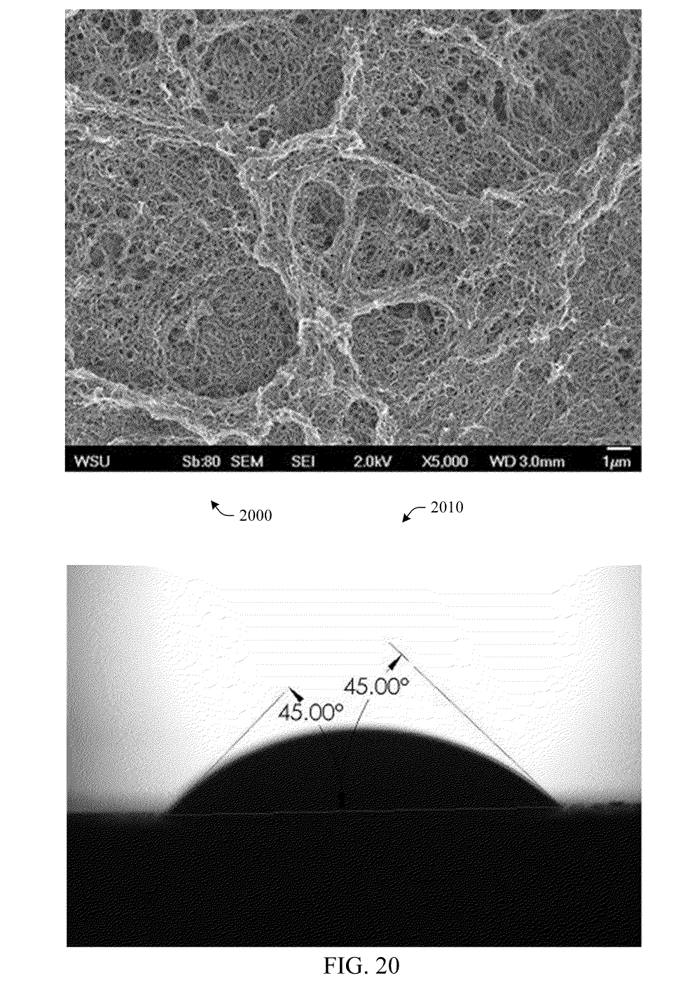

FIG. 20 illustrates the microstructure 2000 and contact angle 2010 of a silica-CNT-HOPG sample from the experiments. Water CA was measured after silica coating on CNT-HOPG and observed to be 45±5.5°, seen in 2010 which was a significant reduction from CA on CNT-HOPG (160°). Strong positive correlation between silica concentration and wettability was also observed, similar to observations made on conventional sol-gel thin films of silica. Unlike plasma treatments, silica coatings induce permanent wettability. However, any liquid based coating on tall CNT arrays will tend to change its global structure due to collapse of the carpet during water evaporation, as seen in FIG. 2000. However, it does not change the CNT stands or any functionality at the local scale. These silica coated CNT-carbon surfaces enable more efficient functionalization with nanoparticles, as well as and better utilization of available surface area for aqueous reactions.

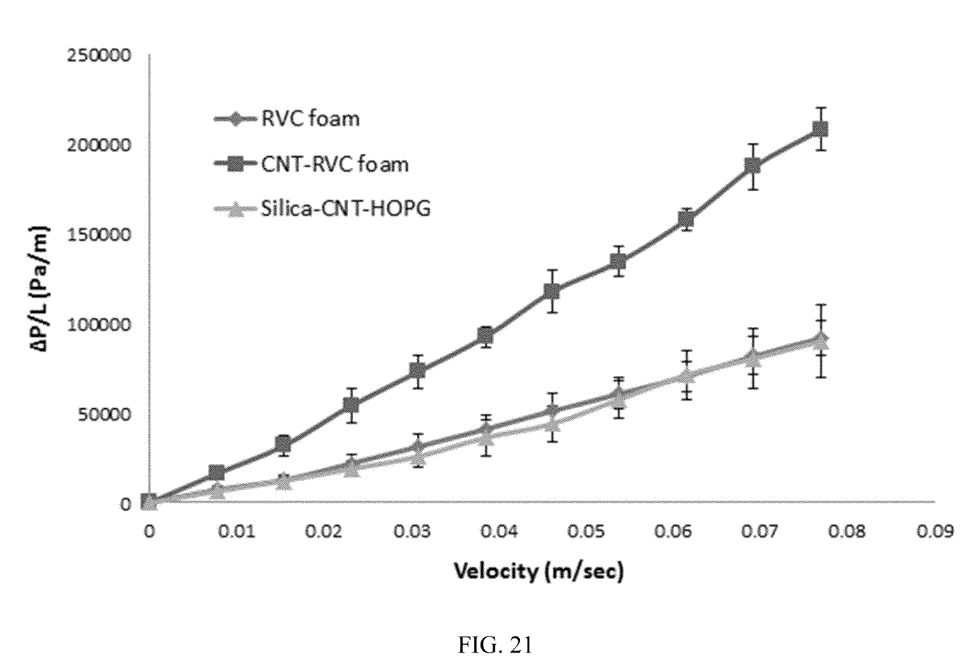

To investigate the influence of wettability on fluid flow characteristics, reticulated carbon foam (RVC foam) was used as a porous substrate suitable for many membrane or filtration related applications. Fluid flow characteristic graphs (Δp/L Vs υ) were measured for three different samples: (1) bare RVC foam, (2) CNT grafted RVC foam (CNT-RVC), and (3) silica coated CNT-RVC (silica-CNT-RVC). FIG. 21 illustrates the average ΔP/L for a given volumetric flux over three samples for each category. The ΔP/L plot indicates the energy required to maintain the given flow rate. The governing equation is:

$$\frac{Δ P}{L} = \frac{α {{S}_{v \text{-} solid}^{2} ( 1 - ɛ )}^{2}}{{ɛ}^{3}} μ ϑ + \frac{β {S}_{v \text{-} solid} ( 1 - ɛ )}{{ɛ}^{3}} ρ {ϑ}^{2} + \frac{{P}_{c}^{e}}{L} \qquad ( 1 )$$

The terms S_{v-solid} and ε are specific surface area and fractional open porosity respectively. α and β are fit coefficients for viscous and inertial terms respectively. (P_{c}^{e}/L) is the “effective capillary pressure” term, which will be negative for hydrophilic surfaces and positive for hydrophobic surface. Some of the governing parameters that can directly influence the ΔP/L are geometrical features of the porous media such as porosity (ε), specific surface area at micro-scale level (S), and effective capillary pressure, which is qualitatively related to surface wettability (θ) in this case. For a planar cylindrical surface, this would be directly proportional to cos θ, but more complicated modeling would be required for this type of hierarchical surface. In this experiment, the varying parameters between various structures were ε, S, and θ.

From the figure it can be seen that, at any given υ, ΔP/L increases with CNT grafting and decreases with silica coating or improved wettability. While the experiments did not develop a quantitative estimate in these complex multiscale solids, the general rule is that lower ε (or lower S) results in higher pressure drops. Also, flow through micro channels is dominated by capillaries and fluid slips. It can be clearly seen that more energy is required for the fluid navigate through CNT-RVC foam compared to untreated foam. This is due to reduced porosity caused by CNT grafting, and further reduction in fluid channel due to super hydrophobicity of the surface. However, when these surfaces were coated with silica, apparent fluid flow channel seems to be increased, aided by capillary driven flows leading them to have lower ΔP/L. This experiment shows that the effective surface area increases with CNT attachment and fluid flow through the CNT carpets can be increased with hydrophilic coating. This can have important implications in applications related to flow of water or biological fluids across such surfaces.

In accordance with aspects of the subject innovation, in wettability experiments, two kinds of surface modification techniques were tested on CNT grafted carbon surfaces: (1) plasma oxygen etching via microwave plasma, and (2) silica coatings via liquid based sol gel method. The oxygen plasma technique improved the surface wettability “temporarily” due to the absorption of hydroxyl groups (C—O/OH). However, hydrophobicity of the surface could be restored due to desorption of C—O/OH groups with application of heat. While switchable wettability on CNT grafted surfaces can have uses in self-cleaning, liquid separation devices, applications requiring permanent wettability can employ alternative techniques. Sol-gel based silica coatings are more suitable for that. Microstructure and crystallographic studies showed the silica coatings to be amorphous. Surface chemistry studies showed that the coatings are clean with no trace elements and equivalent to pure silicon dioxides with BE of Si 2p (103.9 eV) and O 1s (533 eV). Chemistry of coating process was also studied at different stages of coating process and observed that electronic states of Si and O did not change throughout the process, which enables lower annealing temperatures in future. Water CA results showed that wettability of silica coated CNT-carbon was permanently hydrophilic. Fluid flow tests showed that effective surface area is increased due to the improved wettability. These results have applications in liquid purification devices, NPs functionalization, and structural composites.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A hierarchical structure characterized by ultrahigh surface area comprising:
a solid substrate;
an intermediate layer;
at least one plurality of nanoscale attachments that are strongly bonded to the intermediate layer; and
a functional species coating the at least one plurality of nanoscale attachments.

2. The hierarchical structure of claim 1, wherein the functional species comprises a polar oxide.

3. The hierarchical structure of claim 2, wherein the polar oxide comprises silica.

4. The hierarchical structure of claim 2, wherein the functional species comprises a metal oxide.

5. The hierarchical structure of claim 4, wherein the functional species comprises nanoparticles of catalytic metal such as palladium.

6. The hierarchical structure of claim 4, wherein the functional species comprises nanoparticles of an antibacterial species.

7. The hierarchical structure of claim 1, wherein the oxygen containing species comprises absorbed hydroxyl groups.

8.

9. A fluid purification device comprising the hierarchical structure of claim 1.

10. A sensor comprising the hierarchical structure of claim 1.

11. A charge storage device comprising the hierarchical structure of claim 1.

12. A tissue growth scaffold comprising the hierarchical structure of claim 1.

13. A method of fabricating a hierarchical structure comprising:
selecting and preparing a parent substrate;
modifying the substrate surface to form an intermediate layer;
attaching at least one plurality of nanoscale attachments, wherein the nanoscale attachments are selected from nanotubes, nanoparticles, or combinations thereof, onto the intermediate layer;
wherein the steps of forming the intermediate layer and attaching the nanoscale attachments employs one or more of (a) wet chemistry, (b) chemical vapor deposition, (c) plasma deposition, (d) atomic layer deposition, (e) physical vapor deposition, (f) controlled environment heating, or a combination thereof; and
coating the nanoscale attachments with a functional species.

14. The method of claim 12, wherein coating the nanoscale attachments comprises heating the hierarchical structure in a microwave oxygen plasma.

15. The method of claim 13, wherein coating the nanoscale attachments comprises:
preparing an acid catalyzed oxide gel;
dip coating the hierarchical structure in the oxide gel;
drying the hierarchical substrate; and
annealing the hierarchical substrate.

16. The method of claim 14, wherein annealing the hierarchical substrate comprises annealing the hierarchical substrate in inert air.

17. The method of claim 14, wherein preparing an acid catalyzed oxide gel comprises preparing an acid catalyzed silica gel.

18. The method of claim 16, wherein the silica gel comprises a silica precursor and water in a ratio of between 1:40 and 1:60.

19. The method of claim 14, wherein preparing an acid catalyzed oxide gel comprises preparing an acid catalyzed metal oxide gel.

20. The method of claim 18, wherein the acid catalyzed metal oxide gel comprises an acid catalyzed palladium oxide gel.

21. The method of claim 18, wherein the acid catalyzed metal oxide gel comprises an acid catalyzed mixed metal oxide gel.
